(19) United States
(12) Patent Application Publication
(10) Pub. No.: US 2026/0169005 A1
(43) Pub. Date: Jun. 18, 2026

(54) SELECTION OF NANOBODIES USING SEQUENCE FEATURES

(71) Applicants: ZHEJIANG NANOMAB TECHNOLOGY CENTER CO. LTD., SHAOXING, ZHEJIANG (CN); SHANGHAI CHENGHUANG NANOMAB TECHNOLOGY CO. LTD., Jiading District, Shanghai (CN)

(72) Inventors: Xinhao WANG, Shanghai (CN); Jiaguo LI, Shanghai (CN); Wenfeng XU, Shanghai (CN); Weimin ZHU, Shanghai (CN)

(21) Appl. No.: 19/127,088

(22) PCT Filed: Nov. 2, 2023
(86) PCT No.: PCT/CN2023/129236

§ 371 Date: May 2, 2025

Related U.S. Application Data
(60) Provisional application No. 63/382,104, filed on Nov. 2, 2022.

(51) Int. Cl. G01N 33/68; C07K 16/00

(52) U.S. Cl. CPC G01N 33/6845; C07K 16/00; G01N 33/6818; C07K 2317/569; C07K 2317/92

(57) ABSTRACT

Provided is a method of selecting a camelid nanobody from a library of camelid nanobody sequences collected from B cells from a camelid immunized with an antigen. The method comprises: (a) identifying a camelid nanobody that has at least one of the following features (i) a phenylalanine (F) at position 42 (IMGT numbering); (ii) a short hinge; (iii) two or more cysteines in the nanobody sequence; (iv) a glutamine (Q) at position 123 (IMGT numbering); (v) low immunogenicity metric; (vi) non-classic VHH derived from germline IGHV3 or a valine (V) at position 42 (IMGT numbering); (vii) non-classic VHH derived from germline IGHV4 or an isoleucine (I) at position 42 (IMGT numbering); (viii) a histidine (H), aspartic acid (D) or glutamic acid (E) in the CDR region; (ix) a histidine (H), aspartic acid (D) or glutamic acid (E) in the first three amino acid residues, the FR2 region, or the first sixteen amino acid residues of the FR3 region of the nanobody sequence; (x) a tyrosine (Y) at position 42 (IMGT numbering), and the nanobody having a loop, concave paratope structure configuration; or (xi) a phenylalanine (F) at position 42 (IMGT numbering), and the nanobody having a convex paratope structure configuration; and (b) measuring one or more biological activities of the nanobody identified in step (a).

### CROSS-REFERENCE TO RELATED APPLICATIONS

This application claims the benefit of U.S. provisional application No. 63/382,104, filed Nov. 2, 2022, which is incorporated by reference in its entirety.

### BACKGROUND OF THE INVENTION

### (1) Field of the Invention

The present application generally relates to the production of nanobodies that bind to antigen targets of interest. More specifically, methods of selecting nanobodies in a genetic library of nanobody sequences.

### (2) Description of the Related Art

Targeting functional epitopes of a disease target for therapeutics is a big challenge with current antibody technologies because each target has hundreds or thousands of epitopes and only a very limited number of epitopes among them are involved in the biological function. However, current technologies are taking approaches to generate binders randomly, sporadically and experimentally, thus, inadequate coverage of epitopes, redundant selection and low successful rate are the bottleneck.

Camels (Camelus dromedarius and C. bactrianus) belong to old world Camelidae, while llama and alpaca belong to new world Camelidae. There are multiple mechanisms for B-cell primary repertoire diversification of camelids, including preferential usage of the germline V-gene segments, VDJ arrangement, antigen-independent somatic hypermutation (SHM) and gene-conversion-like event, extended hypervariable CDR1 region, non-canonical cysteines and others. Only Camelidae spp. (common name Camelid) have a dichotomous adaptive humoral immune system with both conventional and homodimeric antibodies (HcAb or VHH). In addition, HcAbs have evolved comprehensive paratope architecture as one of the driving factors for recognizing the very wide range of epitopes of the antigen, and IgG1 antibodies complement HcAb binding architecture for more diverse recognitions. Based on current sequence data, bactrian camel has 115 germline VH-gene segments versus 55 VHH-gene segments (Liu et al., 2022), dromedary has 50 VH-gene segments versus 42 VHH-gene segments, while llama has 125 VH-gene segments versus 42 VHH-gene segments (unpublished data), and alpaca has 71 VH-gene segments versus 17 VHH-gene segments. Obviously, the diversification of camelids primary B-cell repertoire is built up more than variation of V-gene germlines.

Camelids have a unique humoral immune system consisting of 2 types of HcAb, IgG2 and IgG3 with long and short hinge regions. Phylogenetic analyses have confirmed that HcAbs diverged from a conventional antibody, IgG1 as a result of recent adaptive changes. It was reported that IgG1 and IgG3 neutralize West Nile virus, whereas IgG2 seems much less effective in an infected or vaccinated animal (Daley et al., 2010). Since this type of viral neutralization may be involved in the Fc region, the better neutralization activity of IgG 3 with short hinge is probably due to its structure conformation. It was also reported that the neutralizing VHHs to TNF-alpha tend to have short hinge type (David R. Maass et al., 2007).

Furthermore, the range of epitopes sampled by HcAb and IgG1 can overlap, but HcAb can also reach sites inaccessible to IgG1. Understanding of the exact roles and functions of the various camelids IgG isotypes is still in its infancy. However, the diverse paratope architecture such as prolate, convex, concave, protrude, and flat surfaces of HcAb (IgG2 and IgG3) offer a great opportunity to develop antibody to challenging targets, especially for diagnostic and therapeutic applications. The simplicity of HcAb without light chain pairing also makes gene cloning and antibody engineering much easier. Furthermore, the conventional IgG1 contributing to 25-75% of total IgG of camelids plays important role to expand an antigen-binding repertoire since the HcAb repertoire of an immunized dromedary or llama displays a recognition pattern that is different from that of conventional IgG1 (McCoy et al., 2012), and certain unique epitopes or druggable target hotspots are accessible to IgG1 with high affinity and desired functionality (Basilico et al. 2014; der Woninga et al., 2016). Camelids have two types of light chains (Vκ or Vλ pairing with VH1 to form conventional IgG1) and their germline organizations have been revealed recently (Griffin et al., 2014; Klarenbeek et al., 2015).

Extensive somatic hypermutation and potential gene conversion are significantly higher among the VHHs than among the VHs (30% versus 1.5%) in the primary VHH B-cell repertoire, which supports further diversification of HcAb repertoire to compensate the lack of light chain.

Equally importantly, the VHH domain of HcAb enlarges the overall antigen-binding repertoire, for example by creating prolate (rugby ball-shaped) structure with a convex paratope surface, which makes it extremely suitable to insert in cavities or clefts (such as active and allosteric sites) on the surface of the antigen. In contrast, the VH-VL domain of conventional IgG contains a more flat or concave paratope surface. The following mechanisms of B-cell repertoire diversification largely contribute to the unique binding characteristics of VHH: (i) most of VHH contains the Framework Region 2 (FR2) with hydrophilic amino acid substitutions comparing to conventional FR2 (V42>F/Y, G49>E/K, L50>R/C, W52>G/L; IMGT numbering, Akhila Melarkode Vattekatte et al., 2020), which participates in the light chain binding; (ii) extended CDR1 region with extensive somatic hypermutation in immune B-cells in residues 27-30 according to Kabat's numbering; (iii) extra disulfide bonds between CDR1-CDR3 (camels) or FR2-CDR3 (llama and alpaca) in large portion of VHH; (iv) extra disulfide bonds within CDR1 and CDR3 in certain portion of VHH; and (v) longer CDR3 loop is also identified possibly due to additional non-templated nucleotide insertions in some VHH (Arbabi-Ghahroudi, 2017; Nguyen et al., 2000; Nguyen et al., 2002; Conrath et al, 2003).

In addition to VHHs (classical VHHs) with FR2 hallmark residues, it was found that there are sets of non-classical VHH (without FR2 hydrophilic amino acids) which are derived from the same gene locus, IGHV3 or IGHV4, D and J as conventional IgG1 do. These VH-like single domains (also called non-classic VHH3 and non-classic VHH4) with an IGHV3 or IGHV4 imprints contain a conventional FR2 GLEW motif and account for approximately 10% and more of total HCAb. Interestingly, different from non-classic IGHV3 HCAbs, IGHV4 gene without the Trp103 substitution can be joined to both sets of C genes to produce classical Abs or to produce HCAbs. In addition, no major difference in sequence or loop structure was discerned between the IGHV4 from classical Abs and HCAbs. It is therefore conceivable that for human therapy one would prefer to select specifically IGHV4-derived HCAbs, non-classic VHH4 instead of IGHV3-based HCAbs or non-classic VHH3 because the latter might require more drastic humanization efforts to minimize immunogenicity (Nick Deschacht, et al., 2010). Overall, these non-classic VHH nanobodies offer a great advantage over classic VHH nanobodies as therapeutic leads because the lack of VHH-featured hydrophilic amino acids (F/Y42, E/K49, R/C50, and G/L52) may greatly reduce immunogenicity risk, which remains to be verified in clinic. In addition, these non-classic VHHs derived from IGHV3 and IGHV4 also likely recognize the same or similar epitopes as IgG1 since both categories of antibodies share the same or similar CDR3 that is responsible for epitope recognition, which expands the pool of antibody leads and provides unique opportunity for developing antibody pairs (Conrath et al., 2003; Deschacht et al., 2010). See also PCT Patent Publication WO 2020/176815.

Furthermore, it was discovered that a small proportion of HCAb sequences (˜0.5-4% of the repertoire) was missing the entire hinge exon, with direct splicing of the VHH and CH2 exons. These hingeless IgG2 and IgG3 HCAbs were distinguishable based on their N-terminal CH2 sequences, however no hingeless conventional IgG1s with directly spliced CH1 and CH2 domains were detected. The bulk of hingeless HCAbs were comprised of a relatively small number of clonally-expanded lineages with unusual properties, including very long CDR-H3s with unusual amino acid content and conventional FR2 GLEW motif. Hingeless HCAb sequences were derived from hinged precursors and showed evidence of SHM, suggesting their potential involvement in antigen-specific immune responses (Kevin A. Henry et al., 2019).

Functional and physical-chemical advantages such as high affinity, specificity, simple gene cloning, high expression yield, ease of purification, highly soluble and stable single-domain fold provide the foundation for HcAb technology. In addition, the antigen-binding repertoire expanded by conventional IgG1 allow even broader epitopes coverage. Furthermore, the close homologies of VHH, VH, Vκ and Vλ to human counterparts offer a great advantage for humanization and therapeutics development.

Nanobodies (VHHs) are used or have potential to be used in many applications with different environmental settings. Such differences may require nanobodies with very different biophysical-chemical properties, including binding affinity, kinetic stability, thermostability, solubility, immunogenicity, expression level and aggregation rate etc. As a diagnostic reagent used in high temperature environment, nanobody will need to be very thermal stable. To fulfill the needs as a therapeutic drug, a nanobody must satisfy many criteria such as functionality, immunogenicity, developability etc. The sequence of a nanobody determines its structure and its biophysical-chemical properties. Sequence features extracted from nanobody sequence information have certain correlations with different biophysical-chemical properties. Thus, to discover a nanobody for specific biophysical-chemical properties, the sequence of a nanobody with certain sequence features can be used to prioritize clone selection.

Natural immune-repertoire exhibits a power-law distribution of its clones: high count clones are very few and many different clones have low counts (FIGS. 1A, 1B). Because of such a distribution, traditional screening methods using phage display, hybridoma or B cell panning technologies are not efficient at identifying low count clones with limited sampling depth. Traditional screening methods enabled people to find high affinity binders in 10⁻¹⁵% repertoire space with around 10 plates (˜1000 clones) (FIG. 1A, 1B). Next generation sequencing (NGS) technology, on the other hand, can sequence millions of clones in a cost-effective manner. Its sampling depth is 3 orders of magnitude higher than traditional screening method with 10 plates. With millions of sequences available, sequence features extracted from sequences in combination with other criteria can be used to select clones from NGS data and discover new nanobodies with specific biophysical-chemical properties.

By taking the advantages of camelids unique antibody organizations and NGS technology to capture entire B-cells antibody repertoire, a novel method is described here to generate hundreds or thousands of diverse antibodies to cover broad epitopes of the target with high-resolution, which enables targeting these important and functional epitopes in systematic and rational manners.

### BRIEF SUMMARY OF THE INVENTION

The present invention is based in part on the discovery that certain sequence features of VHH nanobodies affect the physical and biochemical features of the nanobodies to a surprising degree. Specifically, certain antibody isotype, allotype and other sequence or structural features of camelid nanobodies are believed to largely and intrinsically indicate their binding ability and functionality through antigen-driven diversification, maturation and selection as key part of the secondary B-cell repertoire development, which allows selective antibody development in silico at large scale, followed by more efficient and cost effective experimental validation.

Thus, a method is provided for selecting a camelid nanobody from a library of camelid nanobody sequences collected from B cells from a camelid immunized with an antigen. The method comprises:
(a) identifying a camelid nanobody that has at least one of the following features:
   (i) a phenylalanine (F) at position 42 (IMGT numbering);
   (ii) a short hinge;
   (iii) two or more cysteines in the nanobody sequence;
   (iv) a glutamine (Q) at position 123 (IMGT numbering);
   (v) low immunogenicity metric;
   (vi) non-classic VHH derived from germline IGHV3 or a valine (V) at position 42 (IMGT numbering);
   (vii) non-classic VHH derived from germline IGHV4 or an isoleucine (I) at position 42 (IMGT numbering);
   (viii) a histidine (H), aspartic acid (D) or glutamic acid (E) in the CDR region;
   (ix) a histidine (H), aspartic acid (D) or glutamic acid (E) in the first three amino acid residues, the FR2 region, or the first sixteen amino acid residues of the FR3 region of the nanobody sequence;
   (x) a tyrosine (Y) at position 42 (IMGT numbering), and the nanobody having a loop, concave paratope structure configuration; or
   (xi) a phenylalanine (F) at position 42 (IMGT numbering), and the nanobody having a convex paratope structure configuration; and
(b) measuring one or more biological activities of the nanobody identified in step (a).

In certain embodiments, the low immunogenicity metric is measured by high similarity to human germlines using rarity score (85% or more), percentage identity (80% or more), or low 9-mer score (lower than 40). In some embodiments, the selected camelid nanobody has at least 2, 3, 4, 5, 6, 7, 8 or 9 of the features (i) through (xi).

Also provided is a method for generating a binder that binds to one or more nanobodies but does not substantially bind to an antibody having a leucine at position 123 (IMGT numbering) of the antibody sequence. The method comprises generating the binder that targets the FR4 region of an antibody having a glutamine at position 123 (IMGT numbering). In certain embodiment, the binder is an antibody.

### BRIEF DESCRIPTION OF THE SEVERAL VIEWS OF THE DRAWINGS

FIGS. 1A and 1B are graphs showing the distribution of antibody clones by number of sequences, CDR3 sequence length and count.

FIG. 2 is a flow chart showing exemplary steps for next generation sequencing (NGS) based nanobody discovery using sequence features.

FIGS. 3A and 3B are illustrations of structural differences between CDR3s with a Y at position 42 (FIG. 3A) and an F at position 42 (FIG. 3B) of VHHs. FIG. 3C is a graph showing the differences in minimum CB distance between F and Y groups. FIG. 3D is a graph showing differences in root mean square fluctuation (RMSF) of VHH CDR3 regions between F and Y groups.

FIG. 4 is a graph showing the correlation of CDR3 length and number of cysteines in VHHs.

FIG. 5 is a graph showing the relationship between CDR3 length and percentage of CDR3s without a cysteine and with 1 cysteine.

FIG. 6 is a graph showing average serum fractionation results from 6 Alpacas.

FIG. 7 is a graph showing the number of mismatches between VHHs with short or long hinges.

FIG. 8 is a chart showing the camelid unique residues at FR4. Among 7 species shown here, only alpaca, llama and Bactrian have J genes with a Q in that position.

FIG. 9 is a flow chart showing an example of data processing steps for NGS sequences generated by Miseq.

FIG. 10 is a chart showing the binding affinities of MSLN binders at two pH conditions. These binders exhibit different binding affinities at pH 6 versus at pH 7.

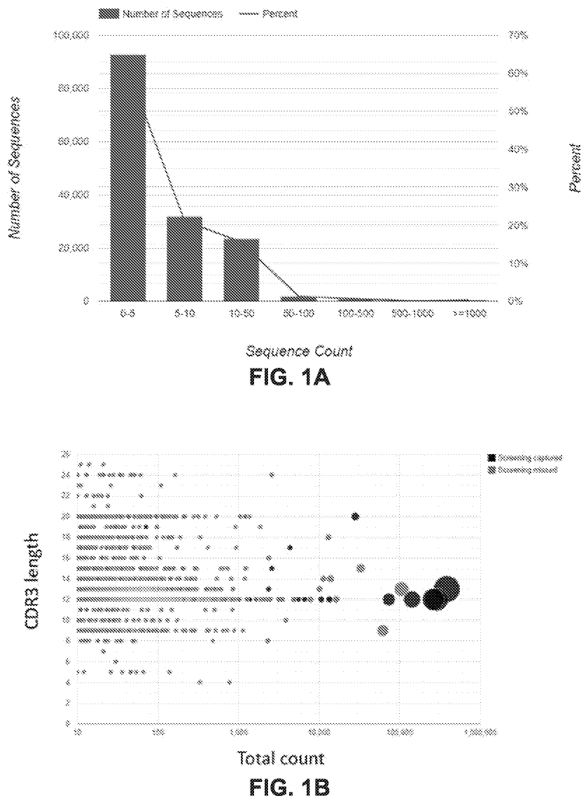

### DETAILED DESCRIPTION OF THE INVENTION

### Definitions

The term “plurality” refers to more than 1, for example more than 2, more than about 5, more than about 10, more than about 20, more than about 50, more than about 100, more than about 200, more than about 500, more than about 1000, more than about 2000, more than about 5000, more than about 10,000, more than about 20,000, more than about 50,000, more than about 100,000, usually no more than about 200,000. A “population” contains a plurality of items.

The term “epitope” as used herein can include any protein determinant capable of specific binding to an immunoglobulin or T-cell receptor. Epitopic determinants usually consist of chemically active surface groupings of molecules such as amino acids or sugar side chains and usually have specific three-dimensional structural characteristics, as well as specific charge characteristics. An antibody is said to specifically bind an antigen when the equilibrium dissociation constant is ≤1 μM, preferably ≤100 nM and most preferably ≤10 nM.

The term “K_{D}” refers to the equilibrium dissociation constant of a particular antibody-antigen interaction.

The term “immune response” as used herein can refer to the action of, for example, lymphocytes, antigen presenting cells, phagocytic cells, granulocytes, and soluble macromolecules produced by the above cells or the liver (including antibodies, cytokines, and complement) that results in selective damage to, destruction of, or elimination from an organism of invading pathogens, cells or tissues infected with pathogens, cancerous cells, or, in cases of autoimmunity or pathological inflammation, normal organismal cells or tissues.

As used herein, the term “antibody” refers to an intact immunoglobulin or to a monoclonal or polyclonal antigen-binding fragment with the Fc (crystallizable fragment) region or FcRn binding fragment of the Fc region, referred to herein as the “Fc fragment” or “Fc region”. Antigen-binding fragments may be produced by recombinant DNA techniques or by enzymatic or chemical cleavage of intact antibodies. Antigen-binding fragments include, inter alia, Fab, Fab′, F(ab′) 2, Fv, dAb, and complementarity determining region (CDR) fragments, single-chain antibodies (scFv), single region antibodies, chimeric antibodies, CDR grafted antibodies, humanized antibodies, biparatopic antibodies, diabodies and polypeptides that contain at least a portion of an immunoglobulin that is sufficient to confer specific antigen binding to the polypeptide. The Fc region includes portions of two heavy chains contributing to two or three classes of the antibody. The Fc region may be produced by recombinant DNA techniques or by enzymatic (e.g. papain cleavage) or via chemical cleavage of intact antibodies.

The term “antibody fragment,” as used herein, refers to a protein fragment that comprises only a portion of an intact antibody, generally including an antigen binding site of the intact antibody and thus retaining the ability to bind antigen. Examples of antibody fragments encompassed by the present definition include: (i) the Fab fragment, having VL, CL, VH and CH1 regions; (ii) the Fab′ fragment, which is a Fab fragment having one or more cysteine residues at the C-terminus of the CH1 region; (iii) the Fd fragment having VH and CH1 regions; (iv) the Fd′ fragment having VH and CH1 regions and one or more cysteine residues at the C-terminus of the CH1 region; (v) the Fv fragment having the VL and VH regions of a single arm of an antibody; (vi) the dAb fragment (Ward et al., 1989) which consists of a VH region; (vii) isolated CDR regions; (viii) F(ab′) 2 fragments, a bivalent fragment including two Fab′ fragments linked by a disulfide bridge at the hinge region; (ix) single chain antibody molecules (e.g., single chain Fv; scFv) (Bird et al., 1988; Huston et al., 1988); (x) “diabodies” with two antigen binding sites, comprising a heavy chain variable region (VH) connected to a light chain variable region (VL) in the same polypeptide chain (see, e.g., EP 404,097; WO 93/11161; Hollinger et al., 1993); (xi) “linear antibodies” comprising a pair of tandem Fd segments (VH-CH1-VH-CH1) which, together with complementary light chain polypeptides, form a pair of antigen binding regions (Zapata et al., 1995; U.S. Pat. No. 5,641,870.

“Single-chain variable fragment”, “single-chain antibody variable fragments” or “scFv” antibodies as used herein refers to forms of antibodies comprising the variable regions of only the heavy (VH) and light (VL) chains, connected by a linker peptide. The scFvs are capable of being expressed as a single chain polypeptide. The scFvs retain the specificity of the intact antibody from which it is derived. The light and heavy chains may be in any order, for example, VH-linker-VL or VL-linker-VH, so long as the specificity of the scFv to the target antigen is retained.

An “isolated antibody”, as used herein, can refer to an antibody that is substantially free of other antibodies having different antigenic specificities (e.g., an isolated antibody that specifically binds a TRAIL protein can be substantially free of antibodies that specifically bind antigens other than TRAIL proteins). An isolated antibody that specifically binds a human TRAIL protein can, however, have cross-reactivity to other antigens, such as TRAIL proteins from other species. Moreover, an isolated antibody can be substantially free of other cellular material and/or chemicals.

The terms “monoclonal antibody” or “monoclonal antibody composition” as used herein can refer to a preparation of antibody molecules of single molecular composition. A monoclonal antibody composition displays a single binding specificity and affinity for a particular epitope.

The term “recombinant human antibody”, as used herein, can refer to all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “isotype” can refer to the antibody class (e.g., IgM or IgG1) that is encoded by the heavy chain constant region genes. An antibody can be an immunoglobulin G (IgG), an IgM, an IgE, an IgA or an IgD molecule, or is derived therefrom.

The term “VHH2”, “VHH3” and “VH1” are representing the heavy chains of three camelid IgG isotypes IgG2, IgG3 and IgG1 respectively. VL1 is representing the light chain of camelid IgG1. Camelid VL′ includes, but not limited to Vκ and Vλ.

The term “correspondingly positioned amino acids” and “corresponding amino acids” used herein interchangeably, are amino acid residues that are at an identical position (i.e., they lie across from each other) When two or more amino acid sequences are aligned. Methods for aligning and numbering antibody sequences are well known in the art.

The term “natural” antibody refers to an antibody in which the heavy and light chains of the antibody have been made and paired by the immune system of a multicellular organism. Spleen, lymph nodes, bone marrow, blood and other lymphatic tissues are examples of tissues that contain cells that produce natural antibodies. For example, the antibodies produced by B cells isolated from a first animal immunized with an antigen are natural antibodies. Natural antibodies contain naturally-paired heavy and light chains.

The term “naturally paired” refers to heavy and light chain sequences that have been paired by the immune system of a multi-cellular organism.

The term “mixture”, as used herein, refers to a combination of elements, e.g., cells, that are interspersed and not in any particular order. A mixture is homogeneous and not spatially separated into its different constituents. Examples of mixtures of elements include a number of different cells that are present in the same aqueous solution in a spatially undressed manner.

The term “assessing” includes any form of measurement, and includes determining if an element is present or not. The terms “determining”, “measuring”, “evaluating”, “assessing” and “assaying” are used interchangeably and may include quantitative and/or qualitative determinations. Assessing may be relative or absolute. “Assessing the presence of” includes determining the amount of something present, and/or determining whether it is present or absent.

The term “enriched” is intended to refer to component of a composition (e.g., a particular type of cells or molecules) that is more concentrated (e.g., at least 2×, at least 5×, at least 10×, at least 50×, at least 100×, at least 500×, at least 1,000×), relative to other components in the sample (e.g., other cells) than prior to enrichment. In some cases, something that is enriched may represent a significant percent (e.g., greater than 2%, greater than 5%, greater than 10%, greater than 20%, greater than 50%, or more, usually up to about 90%-100%) of the sample in which it resides.

The term “enriching” is intended to any way by which antigen-specific cells can be obtained from a larger population of B cells. As described in greater detail below, enriching may be done by panning, using a bead or cell sorting, for example.

The term “obtaining” in the context of obtaining an element, e.g., cells or sequences, is intended to include receiving the element as well as physically producing the element.

The term “peripheral blood mononucleated cells” or “PBMCs” refers to blood cells that have a single approximately round nucleus (as opposed to a lobed nucleus) and includes lymphocytes (T cells, B cells and NK cells), monocytes and macrophage. PBMCs can be enriched from whole blood using a Ficoll gradient.

The term “antigen-specific B cells” refers to memory B cells that have an antibody that specifically binds to an antigen on their surface, as well as progenitors thereof.

A cell is “derived from” a host if the cell, or the progeny thereof, was obtained from the host. The progeny of a progenitor cell is derived from the progenitor cell.

The term “panning” is used to refer to a method by which B cells are applied to a container (e.g., a plate) that has one or more surfaces that are coated in an antigen or portion thereof. Unbound cells can be removed by washing the surface after the cells are applied to it.

The term “bead-based enrichment” is used to refer to a method by which B cells are mixed with beads, e.g., magnetic beads, that are linked to an antigen or portion thereof.

The term “cell sorting” is used to refer to a method by which B cells are mixed a detectable antigen (e.g., a fluorescently detectable antigen) in solution. In cell sorting methods, cells that are bound to the antigen are sorted from the unbound cells. Fluorescence-activated cell sorting (FACS) is an example of a cell sorting method.

The term “activating” is referred to the stimulation of B cells to a) proliferate and b) differentiate into plasma blasts and/or plasma cells and c) secrete antibodies. B cell activation can be done by contacting the B cells with antigen, T cells expressing CD40L and cytokines, although other methods are known (see, e.g., Wykes, Imm. Cell. Biol. 2003 81:328-331).

The term “activated B cells” refers to a cell population that comprises the progeny of a B cell that was activated. As noted above, activation causes B cells to proliferate, and the progeny of such cells are referred to herein as activated B cells.

The term “collecting” refers to the act of separating the cells that in the culture medium from a substrate. Collecting may be done by pipetting or by decanting, for example.

The term “immunized by an antigen” and grammatical equivalents thereof (e.g., “immunized animal”) is intended to refer to any animal (humans, rabbits, mice, rats, sheep, cows, chickens, camels) that is mounting an immune response to an antigen. An animal may be exposed to a foreign antigen via exposure to an infectious agent, a vaccination, or by administrating an antigen and adjuvant (e.g., by injection), for example. The term “immunized by an antigen” is also intended to include animals that are mounting an immune response against a “self” antigen, i.e., have an autoimmune disease.

The term “lineage rank” refers to the order of lineages when they are listed by their priority factors. The priority factors include but not limited to abundancy of lineage sequences, amplification factor, dynamic change of lineage sequence before and after depleting certain unwanted B cells, dynamic change of lineage sequence abundancy during immunization course, lineages which share the same naïve B-cell origin between VHH and VH, avoidance of developability liability sequences and a combination thereof.

The term “hamming distance” refers to the number of positions at which the corresponding symbols are different between two sequences of equal length.

As used herein, the term “grouped antibodies by lineage”, “lineage-related antibodies” and “antibodies that related by lineage” as well as grammatically-equivalent variants thereof, are antibodies that are produced by cells that share a common B cell ancestor. Antibodies that are related by lineage bind to the same epitope of an antigen and are typically very similar in sequence, particularly in their light chain and heavy chain CDR3s. Both the heavy chain and light chain CDR3s of lineage-related antibodies can have an identical length and a near identical sequence (i.e., differ by up to 5, i.e., 0, 1, 2, 3, 4 or 5 residues). Among the group of CDR3s from a lineage, minimal CDR3 distance of a specific CDR3 is the smallest hamming distance of this CDR3 comparing with all other CDR3 of the same length. In some embodiments, the minimal CDR3 distance is equal to or less than 1. In certain cases, the B cell ancestor contains a genome having a rearranged light chain VIC region and a rearranged heavy chain VDJ region, and produces an antibody that has not yet undergone affinity maturation. “Naïve” or “virgin” B cells present in spleen tissue, are exemplary B cell common ancestors.

Related antibodies are related via a common antibody ancestor, e.g., the antibody produced in the naïve B cell ancestor. The term “lineage related antibodies” is intended to describe a group of antibodies that are produced by cells that arise from the same ancestor B-cell. A “lineage group” contains a group of antibodies that are related to one another by lineage.

As used herein, the term “at least the CDR3s” or “at least the CDR3 sequences” refers to only CDR3 sequences, CDR3 sequences in conjunction with CDR1 and/or CDR2 sequences or a sequence of at least 50 contiguous amino acids of the variable domain, up to the entire length of the variable domain, where the sequence contains a CDR3 sequence.

As used herein, the terms “lineage tree” refers to a diagram, resulting from a cladistics analysis, which depicts a hypothetical branching sequence of lineages leading to the individual species of interest. The points of branching within a lineage tree are called nodes.

As used herein, the term “lineage” refers to a theoretical line of descent. Sometimes a group of antibodies related by lineage is referred to as a “lineage group”. The term “lineage” is exclusive, in that a sequence can belong to only one lineage.

As used herein, the term “subgrouping” refers to a further grouping of sequences in a lineage based on unique features or signatures. “Subgroup” is not exclusive, which means one sequence can be in different subgroups. For example, one sequence can have two, three, four, five, or six unique features at the same time. Applying sequence signatures can help to select/narrow-down testing lineages (representative sequences) in a better manner, which may have better biological function/bioactivity outcomes.

As used herein, the term “lineage analysis” refers to the analysis of the theoretical line of descent of an antibody, which is usually done by analyzing a lineage tree.

As used herein, the term “sequence read” refers to a sequence of nucleotides determined by a sequencer, which determination is made, for example, by means of base calling software associated with the technique.

As used herein, the term “obtaining the amino acid sequences” refers to obtaining a file containing amino acid sequences. As is well known, a nucleic acid sequence can be translated into an amino acid sequence in silico.

The term “anchor” and “anchor binder” as used herein interchangeably, is referred to conventional antibody generated with single B-cells sorting or heterohybridoma having native H and L pairing, with that, ones can “position/pair” heavy chain lineage and light chain lineage which consist of a group of sequences derived from clonal expansion of naïve B-cell H and L sequences after encountering the epitope of antigen. Lineages can be “anchored” considering the amino acid sequences of heavy and light chains that are known to pair with one another. In these embodiments, the branches are rotated around their nodes until there is a minimal number of cross-overs (e.g., no crossovers) between the anchored sequences. After the trees have been “aligned” by tanglegram analysis, the leaves that are known to pair can be connected by an edge. If the leaves that are known to pair are connected by an edge, the intervening leaves, in theory, can pair with one another as long as they do not create a cross-over event with an edge or one another.

The phrases “a monoclonal antibody recognizing an epitope on the antigen”, “an antibody recognizing an antigen” and “an antibody specific for an antigen” are used interchangeably herein with the term “an antibody which binds specifically to an antigen” or grammatical equivalents thereof.

The term “specific binding” refers to the ability of an antibody to preferentially bind to a particular antigen that is present in a homogeneous mixture of different molecules. In certain embodiments, a specific binding interaction will discriminate between desirable and undesirable molecules in a sample, in some embodiments more than about 10 to 100 fold or more than e.g., about 1000- or 10,000 fold.

The term “does not substantially bind” to a protein or cells, as used herein, can mean that it cannot bind or does not bind with a high affinity to the protein or cells, i.e., binds to the protein or cells with an K_{D} of 2×10⁻⁶ M or more, more preferably 1×10⁻⁵ M or more, more preferably 1×10⁻⁴ M or more, more preferably 1×10⁻³ M or more, even more preferably 1×10⁻² M or more.

The term “high affinity” for an IgG antibody can refer to an antibody having a K_{D} of 1×10⁻⁶ M or less, preferably 1×10⁻⁷ M or less, more preferably 1×10⁻⁸ M or less, even more preferably 1×10⁻⁹ M or less, even more preferably 1×10⁻¹⁰M or less for a target antigen. However, “high affinity” binding can vary for other antibody isotypes.

The term “pH sensitivity” or “pH responsive” can refer to a binding property of an antibody that shows different binding affinity in different pH environments.

The term “rarity score” can refer to a measure of similarity to human germline sequences. In some embodiments, the value is calculated based on framework regions of germlines. Before calculation, a profile of each residue usage percentage in each length of 4 framework regions is determined based on all human IGHV germlines. For each VHH sequence, the residue in each position of 4 framework regions is compared to the profile of that framework region of same length and the rarity score for each position of framework region is calculated based on usage percentage of that residue divided by the top usage percentage of same position. The rarity score for a sequence is average of rarity scores of all framework region residues.

The term “mismatch score” can refer to the measurement for determining SHM rate. It is calculated as average number of mismatches in 100 bp alignment with the best matched germline gene.

The term “9-mer score” can refer to a measure of similarity between VHH sequences and human antibody sequences found in human immune repertoires and with considerations of natural secreted proteins in human body and Tregitopes. The 9-mer score value can be calculated by traversing the VHH sequences in sliding windows of 9-mer length and computes the score of 9-mer peptide. Subsequently, the scores from individual 9-mers are then averaged to generate an overall score, which provides a quantitative measure of predicted VHH immunogenicity. To calculate the score of each 9-mer peptide, three sets of datasets were established: 9-mer prevalences (percentage of human subjects have 9-mer sequence in repertoire) in human repertoire based on OAS (Observed Antibody Space, https://opig.stats.ox.ac.uk/webapps/oas/) database; Tregitope sequences and secreted human protein sequences curated from public domain. The score of each 9-mer peptide is calculated as follows: if 9-mer sequences in Tregitope sequences, the score is-1; if 9-mer sequences in secreted human protein sequences, the score is 0; otherwise the score is 1-prevalence. A “CDR grafted antibody” is an antibody comprising one or more CDRs derived from an antibody of a particular species or isotype and the framework of another antibody of the same or different species or isotype.

A “humanized antibody” has a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject. In one embodiment, certain amino acids in the framework and constant regions of the heavy and/or light chains of the non-human species antibody are mutated to produce the humanized antibody. In another embodiment, the constant region(s) from a human antibody are fused to the variable region(s) of a non-human species. In another embodiment, a humanized antibody is a CDR grafted antibody comprising one or more CDRs derived from an antibody of a particular species or isotype and the framework of human antibodies. In another embodiment, one or more amino acid residues in one or more CDR sequences of a non-human antibody are changed to reduce the likely immunogenicity of the non-human antibody when it is administered to a human subject, wherein the changed amino acid residues either are not critical for immunospecific binding of the antibody to its antigen, or the changes to the amino acid sequence that are made are conservative changes, such that the binding of the humanized antibody to the antigen is not significantly worse than the binding of the non-human antibody to the antigen. Examples of how to make humanized antibodies may be found in U.S. Pat. Nos. 6,054,297, 5,886,152 and 5,877,293.

The term “chimeric antibody” refers to an antibody that contains one or more regions from one antibody and one or more regions from one or more other antibodies. In one embodiment, one or more of the CDRs are derived from a human antibody. In another embodiment, all of the CDRs are derived from a human antibody. In another embodiment, the CDRs from more than one human antibody are mixed and matched in a chimeric antibody. For instance, a chimeric antibody may comprise a CDR1 from the light chain of a first human antibody, a CDR2 and a CDR3 from the light chain of a second human antibody, and the CDRs from the heavy chain from a third antibody. Other combinations are possible.

The term “biparatopic antibody” refers to an antibody binds to two non-overlapping epitopes of an antigen. In some embodiments, the biparatopic antibody comprises heavy chain only VHHs without light chain. In some embodiments, the biparatopic antibody comprises both heavy chain only VHHs and conventional VH1/VL1 pairs. In some embodiments, the biparatopic antibody comprises two conventional VH1/VL1 pairs. In some embodiments, the biparatopic antibody has a first heavy chain and a first light chain from a monoclonal antibody targeting one epitope, and an additional antibody heavy chain and light chain targeting another epitope. In some embodiments, the additional light chain or heavy chain can be different from the first light or heavy chains.

The term SAbDab refers to the Structural antibody database at opig.stats.ox.ac.uk/webapps/sabdab.

The binding of an antibody of the disclosed invention to an antigen can be assessed using one or more techniques well established in the art. For example, in some embodiments, an antibody is tested by ELISA assays, for example using a recombinant antigen protein. Still other suitable binding assays include but are not limited to a flow cytometry assay in which the antibody is reacted with a cell line that expresses the human antigen, such as HEK293 cells. Additionally or alternatively, the binding of the antibody, including the binding kinetics (e.g., K_{D} value) can be tested in BIAcore binding assays, Octet Red96 (Pall) and the like.

The term “single B-cell sorting” refers to the sorting of isolated and separated single B cells based on antigen specificity. Technologies for single-cell separation, isolation, and sorting include but are not limited to: FACS (fluorescent activated cell sorting, e.g. using a fluorescent-tagged antigen to isolate cells that bind the antigen), ISAAC (immunospot array assays on a chip), LCM (laser-capture microdissection), microengraving, and droplet microfluidics.

The term “IMGT numbering” (Lefranc et al., 2005) refers to one numbering scheme of antibodies.

In some embodiments, provided is a method of selecting a camelid nanobody from a library of camelid nanobody sequences collected from B cells from a camelid immunized with an antigen. The method comprises
(a) identifying a camelid nanobody that has at least one of the following features:
   (i) a phenylalanine (F) at position 42 (IMGT numbering);
   (ii) a short hinge;
   (iii) two or more cysteines in the nanobody sequence;
   (iv) a glutamine (Q) at position 123 (IMGT numbering);
   (v) low immunogenicity metric;
   (vi) non-classic VHH derived from germline IGHV3 or a valine (V) at position 42 (IMGT numbering);
   (vii) non-classic VHH derived from germline IGHV4 or an isoleucine (I) at position 42 (IMGT numbering);
   (viii) a histidine (H), aspartic acid (D) or glutamic acid (E) in the CDR region;
   (ix) a histidine (H), aspartic acid (D) or glutamic acid (E) in the first three amino acid residues, the FR2 region, or the first sixteen amino acid residues of the FR3 region of the nanobody sequence;
   (x) a tyrosine (Y) at position 42 (IMGT numbering), and the nanobody having a loop, concave paratope structure configuration; or
   (xi) a phenylalanine (F) at position 42 (IMGT numbering), and the nanobody having a convex paratope structure configuration; and
(b) measuring one or more biological activities of the nanobody identified in step (a).
   The biological activities include binding affinity, specificity, in vitro and in vivo immunogenicity, thermostability, pH sensitivity, and other biophysical-chemical properties routinely used in antibody validation and clinical development.

Some of the above enumerated sequence or structural features are believed to be more particularly related to certain biological activities of nanobodies. In some embodiments, the features of a phenylalanine (F) at position 42 (IMGT numbering), a short hinge, and/or two cysteines within in the nanobody sequence may be preferred features for selecting nanobodies having high binding affinity. In some embodiments, the features of high similarity to human germlines using rarity score or percentage identity, low 9-mer score, non-classic VHH derived from germline IGHV3 or a valine (V) at position 42 (IMGT numbering), and/or non-classic VHH derived from germline IGHV4 or an isoleucine (I) at position 42 (IMGT numbering) may be preferred features for selecting nanobodies having low in vitro or in vivo immunogenicity. In some embodiments, the features of a glutamine (Q) at position 123 (IMGT numbering) and/or four cysteines in the nanobody sequence or two cysteines within CDR3 may be preferred features for selecting nanobodies having high thermostability. In some embodiments, the features of a histidine (H), aspartic acid (D) or glutamic acid (E) in the CDR region, and/or a histidine (H), aspartic acid (D) or glutamic acid (E) in the first three amino acid residues, the FR2 region, or the first sixteen amino acid residues of the FR3 region of the nanobody sequence, may be preferred features for selecting nanobodies having pH 6.0-selectivity versus pH 7.4. In some embodiments, the features of a tyrosine (Y) at position 42, and the nanobody having a loop, concave paratope structure configuration, and/or a phenylalanine (F) at position 42, and the nanobody having a convex paratope structure configuration, may be preferred features for selecting nanobodies having high binding affinity.

For these methods, the library of camelid nanobody sequences can be created by any means now known or later discovered. In some embodiments, the library is sequenced by next generation sequencing (NGS) methods known in the art. Any NGS method can be utilized in these embodiments. See, e.g., Slatko et al. (2018) for an overview of NGS methods.

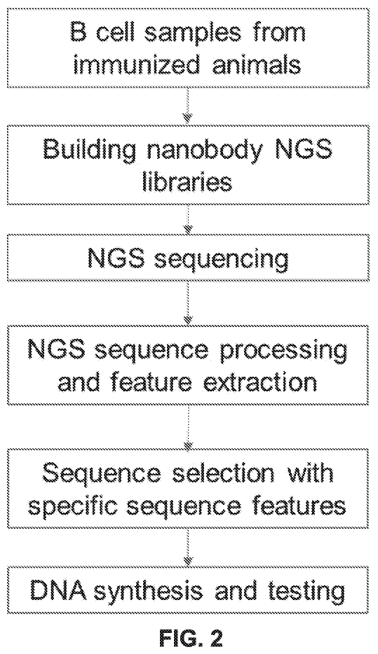

FIG. 2 shows an example of a procedure that can be used in the claimed methods. In these embodiments, NGS libraries for nanobodies are built using B cell samples from immunized animals and NGS sequencing is performed on those libraries. NGS sequences are processed and sequence features for each sequence are extracted. Sequences with specific sequence feature(s) and satisfying other criteria like enrichment score, count etc. are selected. Selected sequences can be synthesized and tested using various functional assays.

In some embodiments, sequences from NGS data are analyzed to identify residue at 42 position (IMGT number). Based on the residue in that position, nanobodies can be classified into four groups (Table 1): F group VHHs with phenylalanine (F) at that position; Y group VHHs with tyrosine (Y) at that position; non-classic VHH3 with valine (V) at that position; and non-classic VHH4 with isoleucine (I) at that position. Such classification is consistent with the results based on the top five germlines matched with classical and non-classical VHH sequences (Table 2).

TABLE 1
Top four residues at position 42 (IMGT
numbering) and corresponding VHH types

| Residue | Percentage | VHH types |
|---|---|---|
| F | 59.0% | F type |
| Y | 26.5% | Y type |
| V | 5.7% | non-classic VHH3 |
| I | 2.5% | non-classic VHH4 |

TABLE 2
Top 5 germlines for classical/non-classical
VHHs and corresponding VHH types

| | | | Residue at | |
|---|---|---|---|---|
| | | | 42 (IMGT | |
| | Germline | Percentage | numbering) | VHH types |
| Classical | IGHV3S65*01 | 29.1% | F | F group |
| | IGHV3-3*01 | 25.8% | F | F group |
| | IGHV3S53*01 | 23.6% | Y | Y group |
| | IGHV3S61*01 | 2.5% | F | F group |
| | IGHV3S66*01 | 2.2% | F | F group |
| Non- | IGHV3S39*01 | 1.5% | V | non-classic |
| classical | | | | VHH3 |
| | IGHV4S5*01 | 1.5% | I | non-classic |
| | | | | VHH4 |
| | IGHV3S42*01 | 1.2% | V | non-classic |
| | | | | VHH3 |
| | IGHV4S1*01 | 0.7% | I | non-classic |
| | | | | VHH4 |
| | IGHV3S41*01 | 0.7% | V | non-classic |
| | | | | VHH3 |

F and Y groups of nanobodies have many distinct differences in biophysical-chemical properties, as summarized in Table 3. A surprising difference between these two groups is the CDR3 length: F group VHHs showed significantly longer CDR3s than Y group VHHs (Table 3). It is known that VHHs have longer CDR3 than conventional VHs. A longer CDR3 compensates for diversity loss due to a lack of light chains.

TABLE 3
Charge and CDR3 length differences between Y vs F group VHHs.

| Position | CDR3 | | CDR net | CDR1 net | CDR2 net | CDR3 net |
|---|---|---|---|---|---|---|
| 42 aa | length** | PI** | charge** | charge** | charge** | charge** |
| Y | 12.03 ± 0.01 | 8.301 ± 0.004 | 0.353 ± 0.005 | 0.113 ± 0.002 | 0.082 ± 0.002 | 0.158 ± 0.004 |
| F | 18.40 ± 0.01 | 7.648 ± 0.003 | −0.145 ± 0.004 | −0.090 ± 0.002 | 0.215 ± 0.002 | −0.271 ± 0.003 |

Numbers are expressed as mean ± standard error,
**(P < 0.0001) indicating significant difference between two groups

The differences in CDR3 length between the VHH and conventional VH appear to be mainly contributed by F group VHHs. In fact, Y group VHHs have a shorter average CDR3 length than those in llama, human or rabbit (VH 12.0 residues in average for Y group VHHs vs 13 for llama VH, 14.86 for rabbit and 15.36 for human). Similar results of Y and F group differences are found in llama and Bactrian camels.

Besides CDR3 length, the other surprising difference between Y and F group VHHs is the charge, as determined at pH 7.4 summing the charges of D (−1), E (−1), R (+1), K (+1) and H (+0.1).

Y group VHHs have significant higher PI value, as determined using the IPC tool at ipc2.mimuw.edu.pl/, CDR net charge, CDR1 and CDR3 net charge than F group VHHs while CDR2 net charge shows the opposite result (Table 3). Overall, Y group VHHs are more positively charged, which is not favorable for antibody specificity (Lilia A. Rabia et al, 2018).

There were also significant differences in hydropathy indices between these two groups (Table 4), as calculated by averaging the hydropathy index of each residue within the region. Y group VHHs show significantly higher hydropathy index (more hydrophobic) than F group VHHs in CDR1 and CDR2, while in CDR3, Y group VHHs show significantly lower hydropathy index than F group VHHs.

TABLE 4
Differences in hydropathy and number of
mismatches between Y vs F group VHHs.

| Posi- | | | | |
|---|---|---|---|---|
| tion | CDR1 | CDR2 | CDR3 | Number of |
| 42 aa | hydropathy** | hydropathy** | hydropathy** | mismatches** |
| Y | 0.242 ± 0.002 | −0.154 ± 0.002 | −0.822 ± 0.001 | 10.93 ± 0.01 |
| F | −0.298 ± 0.001 | −0.357 ± 0.001 | −0.496 ± 0.001 | 9.12 ± 0.01 |

Numbers are expressed as mean ± standard error,
**(P < 0.0001) indicating significant difference between two groups

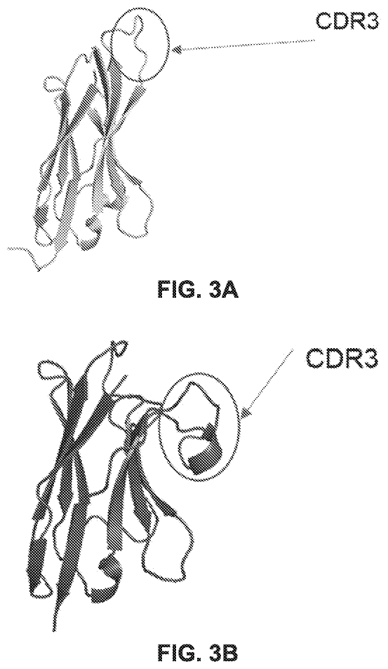

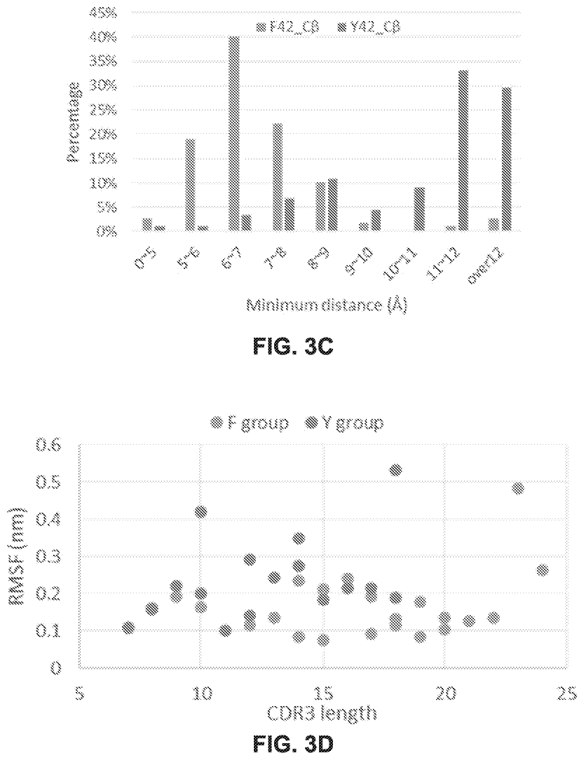

Previous study (Zimmermann et al., 2018) suggested that VHH CDR3s may adopt concave, loop or convex structure configurations. To determine whether there is any connection of such structure configurations with these two groups, VHHs with 3D crystal structures of the two groups were downloaded from SAbDab. Visually, they are very different. Y group VHHs tend to have concave or loop structure configurations (FIG. 3A), while F groups tend to have convex structure configurations which are closer to typical VHH paratope structure configuration (FIG. 3B). One of the main features in convex structures is that CDR3 bends down to form an interaction with FR2. To quantitate the difference, minimum CB distance was analyzed between F or Y residues at position 42 on FR2 and residues on CDR3, without considering the first and last two residues. The result showed surprising differences between these groups (FIG. 3C). Structurally F and Y are similar (below); hydroxylation of F becomes Y. Y is amphipathic while F is hydrophobic, which is probably one main reason why CDR3s with F at position 42 bend down to cover the residue.

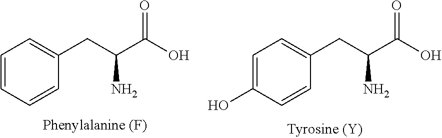 [SMILES: N[C@@H](Cc1ccccc1)C(=O)O ; N[C@@H](Cc1ccc(O)cc1)C(=O)O]

The different CDR3 structure between F and Y group CDR3s may indicate different CDR3 flexibility, which may impact conformational stability, binding affinity, kinetic stability etc. To assess that possibility, several VHH 3D structures were randomly selected from SAbDab for each length of CDR3 of Y and F group VHHs. Excluded from selection were VHHs with a cysteine in the CDR3 region to avoid the impact of disulfide bond on CDR3 flexibility. One hundred ns molecular dynamics (MD) simulations were performed on these VHHs and root mean square fluctuation (RMSF) of CDR3 region was used to assess the flexibility of CDR3 structure. Overall, Y group VHHs showed a significantly higher RMSF value than F group VHHs (P=0.04, T-test, FIG. 3D). If only comparing results with the same range of CDR3 length with 7-18 residues, the difference was even more significant (P=0.01). Such results indicate that the CDR3 in Y group VHHs are more flexible than that in F group.

MD simulations can be performed by any means known in the art. In some embodiments, MD simulations are performed using Gromacs, 2021.2 version, using the following protocols. Briefly, VHH atom coordinates for single chain are extracted from VHH crystal structure PDB files. The VHH structure is placed in a cubic box with a water layer of 0.7 nm using OPLS-AA force field and SPC water. Na+Cl ions are added to neutralize the system. The solvated, electroneutral system is energy minimized. NVT and NPT equilibrations are performed for 100 ps, followed by 100 ns production run at 300 K. The temperature is controlled with a modified Berendsen thermostat and the pressure with an isotropic Parrinello-Rahman at 1 bar.

To assess possible developability differences between the F and Y group VHHs, we measured the retention time of these VHHs in standup monolayer chromatography (SMAC, Kohli, et al., 2015). SMAC measures the non-specific interactions of antibodies with column matrix and its value tends to correlate with antibody precipitation and aggregation. To account for the sequence length differences between different VHHs, we converted retention time to nominal molecular weight using gel filtration standard (Catalog #1511901, www.bio-rad.com) and used the ratio (SMAC ratio) of nominal molecular weight verse theoretical molecular weight to assess the developability of VHHs. Overall, F group VHHs with 2 cysteines have higher SMAC ratio than Y group (T test, P=0.03) or F group with 4 cysteines (T test, P=0.09, Table 5), suggesting that F group VHHs with 2 cysteines have better developability.

TABLE 5
Comparing SMAC ratio between Y and F group VHHs.

| | | F group VHHs with | F group VHHs with |
|---|---|---|---|
| | Y group VHHs | 2 cysteines | 4 cysteines |
| SMAC Ratio | 0.386 ± 0.030 | 0.527 ± 0.061 | 0.397 ± 0.043 |

Numbers are expressed as mean ± standard error

To assess possible functional differences between F and Y group VHHs, binding affinity of VHHs were analyzed by ELISA. Overall, there was no significant difference of normalized ELISA values between Y and F group VHHs (Table 6). However, if only focusing on VHHs without extra disulfide bonds, then F group VHHs had significant higher ELISA value than Y group VHHs (Table 6). F group VHHs with 4 cysteines had significantly lower normalized ELISA values than F group VHHs with 2 cysteines (T-test, P<0.0001) or Y group VHHs (T-test, P=0.01).

TABLE 6
Comparing affinity between Y and F group VHHs.

| Position 42 aa | All VHH | VHH with two cysteines** |
|---|---|---|
| Y | −0.056 ± 0.038 | −0.053 ± 0.040 |
| F | −0.091 ± 0.036 | 0.262 ± 0.079 |

Numbers are expressed as mean ± standard error,
**(P <= 0.0001),
*(P < 0.05) indicating significantly different between two groups

Thus, in some embodiments, the presence or lack of a disulfide bond between the edge of FR2/CDR2 and CDR3 and number of cysteines is determined.

In further embodiments, sequences from NGS data are analyzed to identify the number of cysteines in the sequences. Based on alpaca germlines, VHH germlines are more likely to have extra cysteines (8 out of 17, Table 7) than VH germ lines (7 out of 71). Such result is very similar to camel, indicating the possible important role of extra disulfide bond for VHHs. In NGS dataset we analyzed, 28.5% of VHHs have 4 cysteines. Based on the location of extra cysteines, there are two types of extra disulfide: one between edge of FR2/CDR2 and CDR3 (95.5%) and the other within CDR3 (4.5%). The first type of extra disulfide bond appears to be unique to alpaca and llama, since camels often have an extra disulfide bond between CDR1 and CDR3. Many studies (Govaert et al., 2012, Zabetakis et al., 2014, Kunz et al., 2018) have showed that an extra disulfide bond improves VHH thermostability and conformational stability and reduces aggregation. The higher percentage of extra disulfide bond is considered as another hallmark of VHHs (Govaert et al., 2012, Flajnik et al., 2018).

TABLE 7
17 Alpaca VHH gene lines-positions 39-55

| | | |
|---|---|---|
| AM773729\|IGHV3-3*01 | MGWFRQAPGKEREFVAA | (SEQ ID NO: 1) |
| AM773548\|IGHV3S53*01 | MGWYRQAPGKQRELVAA | (SEQ ID NO: 2) |
| AM939756\|IGHV3S54*01 | MGWYRQAPGKQRELVAA | (SEQ ID NO: 3) |
| AM939763\|IGHV3S55*01 | MGWYRQAPGKERELVAA | (SEQ ID NO: 4) |
| AM939764\|IGHV3S56*01 | MGWYRQAPGKERELVAA | (SEQ ID NO: 5) |
| AM939765\|IGHV3S57*01 | MGWYRQAPGKERELVAA | (SEQ ID NO: 6) |
| AM939752\|IGHV3S58*01 | MGWFRQAPGKEREFVAA | (SEQ ID NO: 7) |
| AM939753\|IGHV3S59*01 | MGWFRQAPGKEREFVAA | (SEQ ID NO: 8) |
| AM939754\|IGHV3S60*01 | MGWFRQAPGKEREFVSC | (SEQ ID NO: 9) |
| AM939757\|IGHV3S61*01 | IGWFRQAPGKEREGVSC | (SEQ ID NO: 10) |
| AM939758\|IGHV3S62*01 | IGWFRQAPGKEREGVSC | (SEQ ID NO: 11) |
| AM939759\|IGHV3S63*01 | IGWFRQAPGKEREGVSC | (SEQ ID NO: 12) |
| AM939760\|IGHV3S64*01 | ISWFRQAPGKEREGVSC | (SEQ ID NO: 13) |
| AM939761\|IGHV3S65*01 | IGWFRQAPGKEREGVSC | (SEQ ID NO: 14) |
| AM939762\|IGHV3S66*01 | IGWFRQAPGKEREGVSC | (SEQ ID NO: 15) |
| AM939766\|IGHV3S67*01 | MSWVRQAPGKERELVAA | (SEQ ID NO: 16) |
| AM939755\|IGHV3S68*01 | MRWFRQAPGKEREWVSC | (SEQ ID NO: 17) |

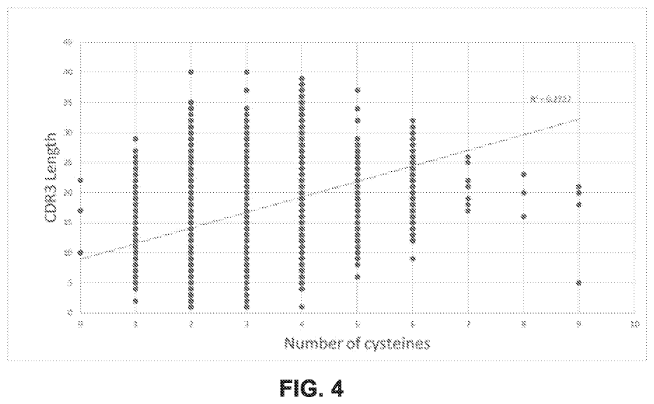

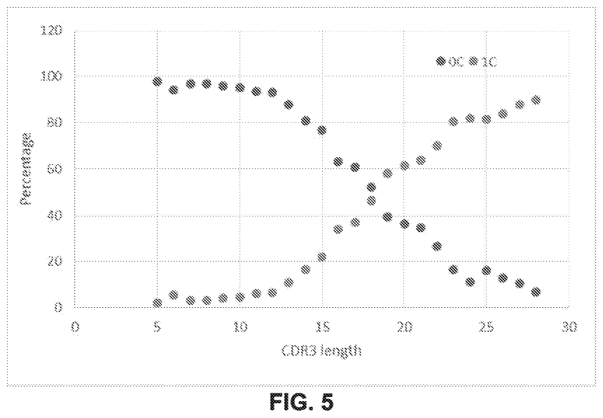

VHHs with longer CDR3 lengths are more likely to have extra disulfide bond. There is a significant positive correlation between number of cysteines and CDR3 length in the whole dataset and in many subgroups (FIG. 4). There are two possible reasons for such results: 1) with longer CDR3 length, it is more likely to have a cysteine residue within the CDR3, either through mutation, or incorporating D genes with a cysteine; and 2) VHH with long CDR3 needs an extra disulfide bond for its conformation stability and functionality. Indeed, a gradual increase of VHHs with one cysteine in CDR3 region started at CDR3 length of 12 residues (FIG. 5). Regarding conformation stability, extra disulfide bond is commonly believed to rigidify and stabilize long CDR3 loops.

In various embodiments, a VHH is identified where CDR1, CDR2 and CDR3 have the same sequences. See WO 2020/176815.

In further embodiments, a VHH is identified where sequences in a lineage map to the same V and J germline genes and where a maximum CDR3 distance of a specific CDR3 is equal or less than 1 between closest two CDR3s from the lineage. See WO 2020/176815.

In additional embodiments, a VHH is identified where sequences in a cluster have the same CDR3 length, where CDR3 identity is greater than 80% between the closest two CDR3s from a cluster. See WO 2020/176815.

In some embodiments, sequences from the library are analyzed to identify hinges in the sequences. Per the IMGT database (www.imgt.org/), publications (Liu et al., 2022; Achour et al., 2008) and our own sequencing efforts, we have summarized hinge sequences for all 4 camelids species as shown in Table 8. Alpaca and llama VHHs use mainly two types of hinges: 2B and 2C while Bactrian and dromedary use 3-4 hinges: 2A, 2C, 3, 3A, and 3B. They shared one common hinge: 2C. Based on the hinge sequence length, hinges can be classified into two groups: short hinge for 2C, 3, 3A and 3B and long hinge for 2A and 2B.

TABLE 8
Camelids nanobody constant genes and corresponding hinge sequences
collected from literature, extracted from genomes and sequenced from
our data

| | Species | Gene (Hinge) | Hinge sequence |
|---|---|---|---|
| New | Alpaca | IGHG2B (2B) | EPKTPKPQPQPQPQ(PQ)PNPTTESKCPKCP |
| World | | | (SEQ ID NO: 18) |
| | | IGHG2C (2C) | AHHSEDPSSKCPKCP |
| | | | (SEQ ID NO: 19) |
| | Llama | IGHG2B (2B) | EPKTPKPQPQPQPQ(PQ)PNPTTESKCPKCP |
| | | | (SEQ ID NO: 20) |
| | | IGHG2C (2C) | AHHSEDPSSKCPKCP |
| | | | (SEQ ID NO: 21) |
| Old | Dromedary | IGHG2A (2A) | EPKIPQPQPKPQPQPQPQPKPQPKPEPECTCPKCP |
| world | | | (SEQ ID NO: 22) |
| | | IGHG2C (2C) | AHHSEDPSSKCPKCP |
| | | | (SEQ ID NO: 23) |
| | | IGHG3 (3) | GTNEVCKCPKCP |
| | | | (SEQ ID NO: 24) |
| | Bactrian | IGHG2A (2A) | EPKIPQPQPKPQPQPQPQPKPQPKPEPECTCPKCP |
| | | | (SEQ ID NO: 25) |
| | | IGHG2C (2C) | AHHPEDPSSQCPKCP |
| | | | (SEQ ID NO: 26) |
| | | IGHG3A (3A) | GTNGGCKCPKCP |
| | | | (SEQ ID NO: 27) |
| | | IGHG3B (3B) | GTNEVCKCPKCP |
| | | | (SEQ ID NO: 28) |

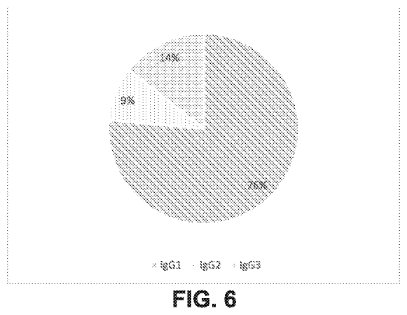

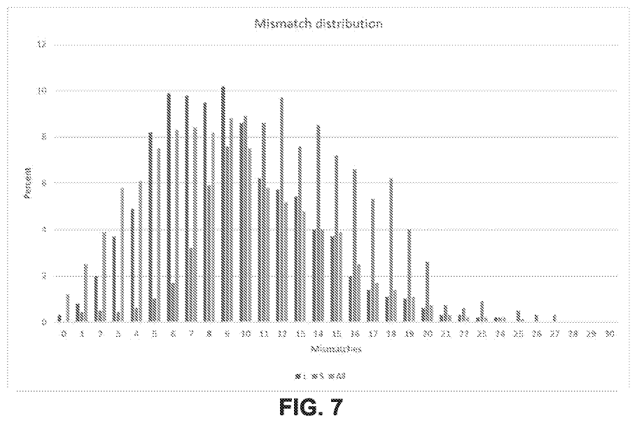

Based on hinges identified in the DNA sequences, VHHs can be grouped into two groups: those with a long hinge (L group, 82.4% of total in alpaca) and those with a short hinge (S group, 17.6% of total in alpaca). FIG. 6 shows a summary of average serum fractionation results of 6 alpacas before panning. S group VHHs in alpaca have significantly higher mismatch values than L group VHHs (FIG. 7), indicating S group VHHs have more somatic mutations. Those SHM were determined by aligning VHH sequences with alpaca germlines downloaded from IMGT using BLAST with similar parameters as used in IgBLAST. The average number of mismatches in 100 bp alignment was used to estimate SHM rate. Similar differences were found in llama and Bactrian (Table 9). However, for bactrian, VHHs only with one of short hinge (3B) showed significantly higher (P<0.0001, T test) mismatch values than VHH with the other three hinge types.

Overall, S group VHHs in alpaca and llama have significantly lower PI, CDR net charge and CDR3 net charge, and higher ELISA binding (Table 9). For bactrian, S group VHHs also have significantly lower PI, CDR and CDR3 net charges as comparing to L group VHHs. The lower CDR net charge and higher ELISA affinity suggest that overall VHHs with short hinges are better therapeutics candidates than VHHs with long hinges.

TABLE 9
Differences between VHHs based on hinges. Numbers
are expressed as mean ± standard error

| Species | Hinge | Mismatches | PI | CDR charge | CDR3 charge | ELISA |
|---|---|---|---|---|---|---|
| Alpaca | 2B | 11.01 ± 0.00 | 7.64 ± 0.00 | −0.22 ± 0.00 | −0.27 ± 0.00 | 1.72 ± 0.02 |
| | 2C | 13.06 ± 0.01 | 7.06 ± 0.00 | −0.77 ± 0.00 | −0.42 ± 0.00 | 2.07 ± 0.03 |
| Llama | 2B | 10.10 ± 0.01 | 7.87 ± 0.00 | −0.04 ± 0.00 | −0.01 ± 0.00 | 1.54 ± 0.02 |
| | 2C | 11.75 ± 0.01 | 7.28 ± 0.00 | −0.68 ± 0.00 | −0.25 ± 0.00 | 1.70 ± 0.05 |
| Bactrian | 2A | 10.58 ± 0.01 | 7.58 ± 0.01 | 0.09 ± 0.01 | 0.31 ± 0.00 | 1.62 ± 0.04 |
| | 2C | 10.93 ± 0.02 | 6.56 ± 0.01 | −1.27 ± 0.01 | −0.56 ± 0.01 | 1.51 ± 0.11 |
| | 3A | 10.59 ± 0.19 | 6.66 ± 0.06 | −1.07 ± 0.09 | −0.40 ± 0.07 | 2.11 ± 0.20 |
| | 3B | 13.93 ± 0.04 | 6.98 ± 0.01 | −0.36 ± 0.02 | −0.04 ± 0.01 | 1.66 ± 0.11 |

In further embodiments, VHHs are analyzed to determine the similarity to human germlines using rarity score or percentage identity and 9-mer score. To use VHHs as therapeutics agents either by itself or in combination with other therapeutic agents in the various formats like antibody drugs, antibody ADC drugs, Car-T etc., immunogenicity of a VHH is a critical issue to consider. Selecting clones with higher homology to human germlines and possibly lower immunogenicity will help to improve the success rate in the downstream drug development.

In other embodiments, sequences from the library are analyzed to identify the residue at position 123 (IMGT number). Based on the residue in that position, nanobodies can be classified into two groups: 1) Q group VHHs with Q at that position, and 2) L group VHHs with L at that position.

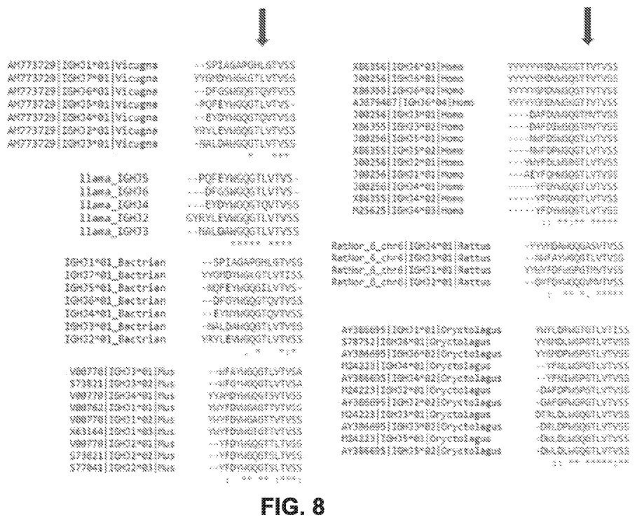

Comparing J genes from 7 species (alpaca, llama, bactrian, human, rat, mouse and rabbit), we found only alpaca (2 out of 7 J genes in IMGT database), llama J genes (1 out of 5 genes in IMGT database) and bactrian (2 out of 7 J genes, identified from its genome sequences) have a Q at position 123 while others only have an L in that position (FIG. 8). A high homology blast search of several camelid genomes showed similar results in other camelid species like Camelus dromedarius. Analyzing all J genes from the IMGT database showed that some fish related species like Danio rerio_Tuebingen, Oncorhynchus mykiss_Swanson and Salmo salar also have a Q at that position.

In our NGS dataset, 89.6% of VHHs from alpaca in NGS data have Q in that position while only 6.7% of VHHs in NGS have L (Table 10). Similar percentages were observed in classical VHH and non-classical VHH groups (Table 10). Even though only 2 out of 7 J genes had a Q residue at that position, close to 90% of VHHs from NGS data have a Q at that position, suggesting an important role of this residue for VHH. Based on this residue, we can group VHHs into Q and L groups. Similar ratios were found in llama (85.7% for Q group vs 9.9% for L group) and in bactrian (87.4% for Q group vs 8.0% for L group).

TABLE 10
Position 123 Q or L percentages in alpaca

| | | Percentage | CDR3 length |
|---|---|---|---|
| NGS | Classical | 89.6% (407028) vs 6.7% | 16.02 vs 16.84** |
| | (Q vs L) | (30230) | |
| | Non-classical | 90.2% (32351) vs 7.8% | 12.56 vs 13.99** |
| | (Q vs L) | (2780) | |
| | All (Q vs L) | 89.6%% (439379) vs 6.7% | 15.77 vs 16.60** |
| | | (33010) | |

Between Q and L group VHHs, a surprising difference is the percentage of extra disulfide bonds. L group VHHs are significantly more likely to have extra disulfide bonds than Q group VHHs. 45.0% of L group VHHs have 4 cysteines (extra disulfide bond) as comparing to only 26.8% of Q group VHHs having 4 cysteines (P<0.0001, Chi-Square test, Table 11). Interestingly, the group of VHHs formed by the overlapping of the F group and L group has the highest percentage of VHHs with 4 cysteines (80.5%) and also has the longest CDR3 length (19.62). It is known that extra disulfide bonds help stabilize VHH structures. Such results suggest that the possible stabilization role of Q resides at position 123. Indeed, previous studies showed the importance of the Q residue for the production efficiency of llama VHHs in Saccharomyces cerevisiae (Gorlani et al., 2012).

TABLE 11
Q vs. L in Position 123 and cysteines

| | | 2 C | | 4 C | |
|---|---|---|---|---|---|
| All** | Q | 284115 | (64.7%) | 117598 | (26.8%) |
| (P < 0.0001) | L | 16283 | (49.3%) | 14838 | (45.0%) |
| Classical** | Q | 254694 | (62.6%) | 116988 | (28.7%) |
| (P < 0.0001) | L | 13730 | (45.4%) | 14790 | (48.9%) |
| Non-classical | Q | 29421 | (90.9%) | 610 | (1.9%) |
| | L | 2553 | (91.8%) | 48 | (1.7%) |

The uniqueness of Q residue in VHH germlines, not in human, mouse, rat or rabbit VH germlines, provides an opportunity to develop VHH specific binding agents. By developing an antibody targeting this specific residue, detection agents can be developed which specifically detect VHH, not human, mouse, rat or rabbit antibodies.

Additionally, antibodies against soluble antigens and shed membrane antigens are usually designed to bind their targets at neutral pH and release the targets at acidic pH. This approach allows efficient elimination of these antigens from bodily fluids through lysosome degradation, but antibody recycle back to circulation to profoundly increase the half-life of antibody. In contrast, targeting membrane-bound antigens associated with solid tumors in tumor micro-environment by acidic pH-selective antibodies, but not in normal tissue environments (pH 7.4) can dramatically reduce the on-target off-tumor cytotoxicity. As we know, pH-responsive antibodies sense pH due to histidine residues within their variable regions. pKa value of the histidine side chain is about 6; thus, at pH below 6.0, the histidine side chain is mostly protonated, whereas, at physiologic pH 7.4, it is deprotonated. It was shown that an increased number of ionizable groups correlates with stronger pH-dependency. Aspartate and glutamate have similar characteristics as those of histidine, but to a less degree.

In other embodiments, VHH sequences from the lineage binding to the desired epitope are analyzed to identify clones containing pH-sensitive amino acids such as histidine, aspartate or glutamate (Peter S. Lee et al. 2022), and an experiment is setup to screen pH-selective VHH at pH 6.0 (tumor microenvironment) versus at pH7.4 (normal physiological condition) (Hwai Wen Chang et al. 2021). Histidine, compared with aspartate and glutamate, is rare within germline and matured sequences of CDRs in antibodies. The NGS library approach described in this application is thus believed to offer an effective screening method for nanobodies that are pH 6.0-sensitive versus pH 7.4, by identifying VHH nanobodies having primarily histidine, and secondarily aspartic acid and glutamic acid.

In various embodiments of these methods, more than one, e.g., 2, 3, 4, 5, 6, 7, 8 or 9, of the enumerated features are identified.

In some embodiments, the low immunogenicity metric is measured by high similarity to human germlines using rarity score or percentage identity, or low 9-mer score.

In specific embodiments, a nanobody with only two cysteines and an F at position 42 is identified.

In specific embodiments, a nanobody having at least one of the following features is identified:
(i) a phenylalanine (F) at position 42 (IMGT numbering);
(ii) a short hinge; or
(iii) two cysteines within in the nanobody sequence;
and the binding affinity of the nanobody is measured.

In specific embodiments, a nanobody having at least one of the following features is identified:
(i) low immunogenicity matric;
(ii) non-classic VHH derived from germline IGHV3 or a valine (V) at position 42 (IMGT numbering); or
(iii) non-classic VHH derived from germline IGHV4 or an isoleucine (I) at position 42 (IMGT numbering);
and the immunogenicity in vitro or in vivo of the nanobody is measured.

In specific embodiments, a nanobody having at least one of the following features is identified:
(i) a glutamine (Q) at position 123 (IMGT numbering); or
(ii) four cysteines in the nanobody sequence or two cysteines in the CDR3;
and thermostability of the nanobody is measured.

In specific embodiments, a nanobody having at least one of the following features is identified:
(i) a histidine (H), aspartic acid (D) or glutamic acid (E) in the CDR region; or
(ii) a histidine (H), aspartic acid (D) or glutamic acid (E) in the first three amino acid residues, the FR2 region, or the first sixteen amino acid residues of the FR3 region of the nanobody sequence;
and the binding affinities of the nanobody at pH 6.0 versus pH 7.4 are measured. Among such nanobodies, those that are pH dependent are selected for further development.

In specific embodiments, a nanobody having at least one of the following features is identified:
(i) a tyrosine (Y) at position 42, and the nanobody having a loop, concave paratope structure configuration; or
(ii) a phenylalanine (F) at position 42, and the nanobody having a convex paratope structure configuration;
   and the binding affinity of the nanobody is measured.

In some embodiments, the library data is processed by:
(i) grouping nanobody sequences in the libraries by CDR sequences (CDR groups), lineages and clusters; and
(ii) generating an enrichment score for sequences, CDR groups, lineages and/or clusters by comparing their frequencies in NGS data after enrichment to those before enrichment or before and after immunization. See, e.g., WO 2020/176815.

In other embodiments, sequences from NGS data are analyzed to identify possible development liabilities, including but not limited to: unpaired cysteine, n-linked glycosylation, methionine oxidation, tryptophan oxidation, asparagine deamidation aspartic acid isomerization, lysine glycation, n-terminal glutamates, integrin binding, CD11c and fragmentation. See, e.g., WO 2020/176815. Such information can be used to further filter out selected clones.

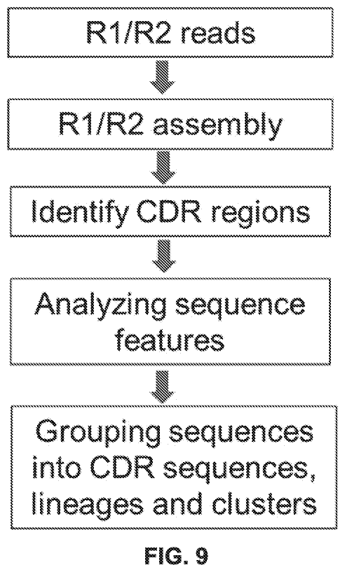

In some embodiments, at least two NGS libraries are constructed: libraries from samples before and after enrichment or from samples before and after immunization. Sequences generated from these libraries are processed to identify CDR regions, germline sequence, count and frequency for each sequence (FIG. 9). An enrichment score for each sequence is generated by comparing the frequency of that sequence between two samples. Sequences are grouped into CDR sequences if their CDR1, CDR2 and CDR3 sequences are identical. Sequences are further grouped into lineages if sequences mapped to same V/J germline genes and having same length of CDR3 with maximum one aa difference with CDR3 length longer than 4 and 0 difference for CDR3 length equal or shorter than 4, and clusters if sequences have same length of CDR3 with 80% or more identity in CDR3 sequences. Similar enrichment scores for groups are also calculated. To improve prediction results, sequences with specific features are further filtered based on enrichment scores in sequences and groups. Clones do not show any enrichment in sequences and groups are excluded from further testing. See, e.g., WO 2020/176815.

In other embodiments, the identifying or selecting procedure is repeated to optimize a sequence within the same lineage group of the selected antibodies. See, e.g., WO 2020/176815.

In additional embodiments, the method further comprises repeating the steps of identifying or selecting a representative sequence from a lineage comprising a VHH2 or VHH3 with a top ranking by lineage priority factors in the NGS library and testing an antibody comprising the selected these sequence to determine if the antibody binds to the antigen or portion thereof to generate camelid antibodies, wherein the representative sequences are selected from top 101-200, 201-300, 301-400, 401-500, 501-600, 601-700, 701-800, 801-900, 901-1000, 1001-1100, 1101-1200, 1201-1300, 1301-1400, 1401-1500, 1501-1600, 1601-1700, 1701-1800, 1801-1900, or 1901-2000 ranking lineages. In some embodiments, the method further comprises repeating the above selecting and testing steps to generate camelid antibodies, wherein the representative sequences are selected from top 2,000 to 10,000 ranking lineages. See, e.g., WO 2020/176815.

The selected nanobody can be expressed by any means known in the art, e.g., in prokaryotic cells or eukaryotic cells.

In various embodiments, the identified nanobody is further characterized, e.g., by measuring binding affinity to the antigen or the specific epitope on the antigen, specificity, in vitro and in vivo immunogenicity, thermostability, pH sensitivity, and other biophysical-chemical properties routinely used in antibody development.

Another method provided in this application is a method for generating a binder that binds to one or more nanobodies but does not substantially bind to an antibody having a leucine at position 123 (IMGT numbering) of the antibody sequence. The method comprises generating the binder that targets the FR4 region of an antibody having a glutamine at position 123 (IMGT numbering). In some embodiments, the antibody having a leucine at position 123 (IMGT numbering) of the antibody sequence is a human antibody, a mouse antibody, or a rabbit antibody. In some embodiments, the binder is an antibody or a binding fragment thereof.

Example: pH sensitive VHH binder discovery by selecting sequences with specific features from NGS data

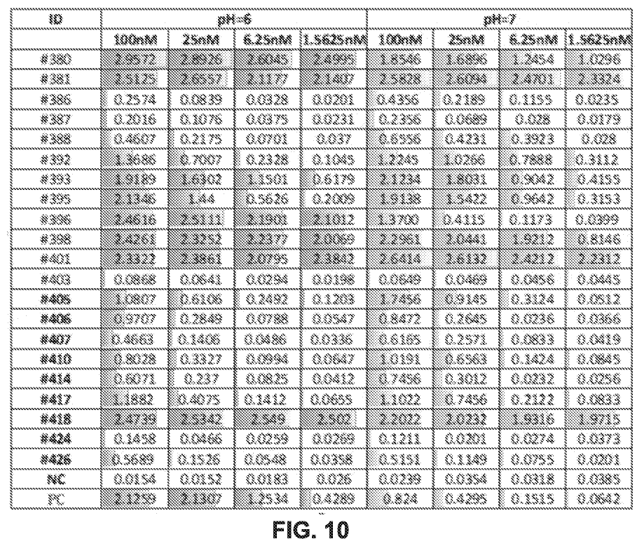

To discover possible pH sensitive binders against MSLN target for CAR-T therapy, we selected 47 sequences from a cluster with known binders. These 47 sequences were selected as they have HIS residues in CDR (CDR1, CDR2 and/or CDR3) region. These sequences were synthesized and expressed. For those expressed, ELISA assays were performed in two pH conditions. As shown in FIG. 10, several clones showed binding affinity differences between the two pH conditions. Further validation confirmed that #396 consistently demonstrated pH sensitive binding: higher binding affinity with pH at 6 and lower binding affinity with pH at 7 or 7.4.

### Embodiments

Embodiment 1. A method of selecting a camelid nanobody from a library of camelid nanobody sequences collected from B cells from a camelid immunized with an antigen, the method comprising
(a) identifying a camelid nanobody that has at least one of the following features
   (i) a phenylalanine (F) at position 42 (IMGT numbering);
   (ii) a short hinge;
   (iii) two or more cysteines in the nanobody sequence;
   (iv) a glutamine (Q) at position 123 (IMGT numbering);
   (v) low immunogenicity metric;
   (vi) non-classic VHH derived from germline IGHV3 or a valine (V) at position 42
   (IMGT numbering);
   (vii) non-classic VHH derived from germline IGHV4 or an isoleucine (I) at position 42 (IMGT numbering);
   (viii) a histidine (H), aspartic acid (D) or glutamic acid (E) in the CDR region;
   (ix) a histidine (H), aspartic acid (D) or glutamic acid (E) in the first three amino acid residues, the FR2 region, or the first sixteen amino acid residues of the FR3 region of the nanobody sequence;
   (x) a tyrosine (Y) at position 42 (IMGT numbering), and the nanobody having a loop, concave paratope structure configuration; or
   (xi) a phenylalanine (F) at position 42 (IMGT numbering), and the nanobody having a convex paratope structure configuration; and
   (b) measuring one or more biological activities of the nanobody identified in step (a).

Embodiment 2. The method of embodiment 1, wherein the low immunogenicity metric is measured by high similarity to human germlines using rarity score or percentage identity, or low 9-mer score.

Embodiment 3. The method of embodiment 1 or 2, wherein the selected nanobody has at least 2, 3, 4, 5, 6, 7, 8 or 9 of the features.

Embodiment 4. The method of any one of embodiments 1-3, wherein the biological activities comprise binding affinity, immunogenicity, thermostability, and pH sensitivity.

Embodiment 5. The method of any one of embodiments 1-4, wherein a nanobody with only two cysteines and an F at position 42 is selected.

Embodiment 6. The method of any one of embodiments 1-4, wherein the camelid nanobody in step (a) has at least one of the following features:
(i) a phenylalanine (F) at position 42 (IMGT numbering);
(ii) a short hinge; or
(iii) two cysteines within in the nanobody sequence; and
wherein the biological activity in step (b) is binding affinity.

Embodiment 7. The method of any one of embodiments 1-4, wherein the camelid nanobody in step (a) has at least one of the following features:
(i) low immunogenicity metric;
(ii) non-classic VHH derived from germline IGHV3 or a valine (V) at position 42 (IMGT numbering); or
(iii) non-classic VHH derived from germline IGHV4 or an isoleucine (I) at position 42 (IMGT numbering); and
wherein the biological activity in step (b) is immunogenicity in vitro or in vivo.

Embodiment 8. The method of any one of embodiments 1-4, wherein the camelid nanobody in step (a) has at least one of the following features:
(i) a glutamine (Q) at position 123 (IMGT numbering); or
(ii) four cysteines in the nanobody sequence; and wherein the biological activity in step (b) is thermostability.

Embodiment 9. The method of any one of embodiments 1-4, wherein the camelid nanobody in step (a) has at least one of the following features:
(i) a histidine (H), aspartic acid (D) or glutamic acid (E) in the CDR region;
(ii) a histidine (H), aspartic acid (D) or glutamic acid (E) in the first three amino acid residues, the FR2 region, or the first sixteen amino acid residues of the FR3 region of the nanobody sequence; and wherein the biological activity in step (b) is binding affinity at pH 6.0 versus pH 7.4, and wherein the nanobodies that are pH dependent are selected.

Embodiment 10. The method of any one of embodiments 1-4, wherein the camelid nanobody in step (a) has at least one of the following features:
(i) a tyrosine (Y) at position 42, and the nanobody having a loop, concave paratope structure configuration; or
(ii) a phenylalanine (F) at position 42, and the nanobody having a convex paratope structure configuration; and wherein the biological activity in step (b) is binding affinity.

Embodiment 11. The method of any one of embodiments 1-10, wherein the library is an NGS library.

Embodiment 12. The method of embodiment 11, wherein the library data is processed by:
(a) grouping nanobody sequences in the libraries by CDR sequences (CDR groups), lineages and clusters; and
(b) generating an enrichment score for sequences, CDR groups, lineages and/or clusters by comparing their frequencies in NGS data after enrichment to those before enrichment or before and after immunization.

Embodiment 13. The method of any one of embodiments 1-12, wherein the selection procedure is repeated to optimize a sequence within the same lineage group of the selected antibodies.

Embodiment 14. The method of any one of embodiments 1-13, wherein the identifying procedure is repeated to optimize a sequence within the same lineage group of the selected antibodies.

Embodiment 15. The method of any one of embodiments 1-14, wherein the selected nanobody is expressed in prokaryotic cells.

Embodiment 16. The method of any one of embodiments 1-14, wherein the selected nanobody is expressed in eukaryotic cells.

Embodiment 17. The method of any one of embodiments 1-16, wherein binding affinity of the selected nanobody to the antigen is measured.

Embodiment 18. The method of any one of embodiments 1-17, wherein binding affinity of the selected nanobody to an epitope of the antigen is measured.

Embodiment 19. A method for generating a binder, wherein the binder binds to one or more nanobodies but does not substantially bind to an antibody having a leucine at position 123 (IMGT numbering) of the antibody sequence, comprising generating the binder that targets the FR4 region of an antibody having a glutamine at position 123 (IMGT numbering).

Embodiment 20. The method of embodiment 19, wherein the antibody having a leucine at position 123 (IMGT numbering) of the antibody sequence is a human antibody, a mouse antibody, or a rabbit antibody.

Embodiment 21. The method of embodiment 19 or 20, wherein the binder is an antibody or a binding fragment thereof.

In view of the above, it will be seen that several objectives of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification, including but not limited to patent publications and non-patent literature, and references cited therein, are hereby incorporated by reference. The discussion of the references herein is intended merely to summarize the assertions made by the authors and no admission is made that any reference constitutes prior art. Applicants reserve the right to challenge the accuracy and pertinence of the cited references.

As used herein, in particular embodiments, the terms “about” or “approximately” when preceding a numerical value indicates the value plus or minus a range of 10%. Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. That the upper and lower limits of these smaller ranges can independently be included in the smaller ranges is also encompassed within the disclosure, subject to any specifically excluded limit in the stated range. Where the stated range includes one or both of the limits, ranges excluding either or both of those included limits are also included in the disclosure.

The indefinite articles “a” and “an,” as used herein in the specification and in the embodiments, unless clearly indicated to the contrary, should be understood to mean “at least one.”

The phrase “and/or,” as used herein in the specification and in the embodiments, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements can optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the embodiments, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the embodiments, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the embodiments, shall have its ordinary meaning as used in the field of patent law.

As used herein in the specification and in the embodiments, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements can optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

### REFERENCES

Achour, I., Cavelier, P., Tichit, M., Bouchier, C., Lafaye, P., & Rougeon, F. (2008). Tetrameric and Homodimeric Camelid IgGs Originate from the Same IgH Locus. The Journal of Immunology, 181 (3), 2001-2009.
Akhila Melarkode Vattekatte et al., Discrete analysis of camelid variable domains: sequences, structures, and in-silico structure prediction. PeerJ. 8: e8408, 2020.
Arbabi-Ghahroudi. Camelid Single-Domain Antibodies: Historical Perspective and Future Outlook. Front. Immunol., 20 Nov. 2017.
Basilico, et al. Four individually druggable MET hotspots mediate HGF-driven tumor progression, The Journal of Clinical Investigation, Volume 124 Number 7 Jul. 2014.
Bird et al., Science 242:423-426 (1988).
Conrath K E, et al. Emergence and evolution of functional heavy-chain antibodies in Camelidae. Dev Comp Immunol 27:87-103, 2003.
Daley L P, Clin. Vaccine Immunol. 17:239-46, 2010.
David R. Maass et al., Alpaca (Lama pacos) as a convenient source of recombinant camelid heavy chain antibodies (VHHs). J Immunol Methods. 2007 Jul. 31; 324 (1-2): 13-25.
Deschacht, et al. A Novel Promiscuous Class of Camelid Single-Domain Antibody Contributes to the Antigen-Binding Repertoire, The Journal of Immunology. 184 (10) 5696-5704 May 2010.
Flajnik M F, Deschacht N, Muyldermans S. A Case Of Convergence: Why Did a Simple Alternative to Canonical Antibodies Arise in Sharks and Camels? (2011) doi: 10.1371/journal.pbio.1001120.
Griffin et al. Analysis of heavy and light chain sequences of conventional camelid antibodies from Camelus dromedarius and Camelus bactrianus species, Journal of Immunological Methods Volume 405, Pages 35-46, March 2014.
Gorlani A, Lutje Hulsik D, Adams H, Vriend G, Hermans P, Verrips T. Antibody engineering reveals the important role of J segments in the production efficiency of llama single-domain antibodies in Saccharomyces cerevisiae. Protein Eng Des Sel (2012) 25:39-46.
Govaert J, Pellis M, Deschacht N, Vincke C, Conrath K, Muyldermans S, Saerens D.
Dual beneficial effect of interloop disulfide bond for single domain antibody fragments. J Biol Chem (2012) 287:1970-1979. doi: 10.1074/jbc.M111.242818.
Hollinger et al., Proc. Natl. Acad. Sci. USA, 90:6444-6448 (1993).
Huston et al., PNAS (USA) 85:5879-5883 (1988).
Hwai Wen Chang et al. Generating tumor-selective conditionally active biologic anti-CTLA4 antibodies via protein-associated chemical switches, PNAS 2021 Vol. 118 No. 9 e2020606118.
Lefranc, M. P., Pommié, C., Kaas, Q., Duprat, E., Bosc, N., Guiraudou, D., Jean, C., Ruiz, M., da Piédade, I., Rouard, M., Foulquier, E., Thouvenin, V., & Lefranc, G. (2005). IMGT unique numbering for immunoglobulin and T cell receptor constant domains and Ig superfamily C-like domains. Developmental and Comparative Immunology, 29 (3), 185-203.
Lilia A. Rabia et al, Net charge of antibody complementarity determining regions is a key predictor of Specificity, Protein Engineering, Design & Selection, 2018, vol. 31 no. 11, pp. 409-418.
Liu, Y. et al., Characterization of heavy-chain antibody gene repertoires in Bactrian camels. Journal of Genetics and Genomics 2022. https://doi.org/10.1016/j.jgg.2022.04.010.
Kevin A. Henry et al., Llama peripheral B-cell populations producing conventional and heavy chain-only IgG subtypes are phenotypically indistinguishable but immunogenetically distinct. Immunogenetics, 2019 April; 71 (4): 307-320.
Klarenbeek, et al. Camelid Ig V genes reveal significant human homology not seen in therapeutic target genes, providing for a powerful therapeutic antibody platform, mAbs 7:4, 693-706; 2015.
Kohli N, Jain N, Geddie M L, Razlog M, Xu L, Lugovskoy A A. A novel screening method to assess developability of antibody-like molecules. MAbs. 2015; 7 (4): 752-8.
Kunz P, Zinner K, Mücke N, Bartoschik T, Muyldermans S, Hoheisel J D. The structural basis of nanobody unfolding reversibility and thermoresistance. Sci Rep (2018) 8:1-10. doi: 10.1038/s41598-018-26338-z.
McCoy L E, et al. Potent and broad neutralization of HIV-1 by a llama antibody elicited by immunization. J. Exp. Med. 2012.
Nick Deschacht, et al., A Novel Promiscuous Class of Camelid Single-Domain Antibody Contributes to the Antigen-Binding Repertoire. The Journal of Immunology, May 15, 2010, 184 (10) 5696-570.
Nguyen et al. Camel heavy-chain antibodies: diverse germline VHH and specific mechanism enlarge the antigen-binding repertoire The EMBO Journal 19 No. 5 2000.
Nguyen et al. Heavy-chain antibodies in Camelidae; a case of evolutionary innovation. Immunogenetics 54:39-47, 2002.
Peter S. Lee et al. Improved therapeutic index of an acidic pH selective antibody, mAbs, 2022, VOL. 14, NO. 1, e2024642.
Slatko et al, Curr. Protoc. Mol. Biol. 122: e59, 2018.
Ward et al., Nature 341, 544-546 (1989).
Woninga, et al. DNA immunization combined with scFv phage display identifies antagonistic GCGR specific antibodies and reveals new epitopes on the small extracellular loops, MABS, VOL. 8, NO. 6, 1126-1135, 2016.
Zabetakis D, Olson M A, Anderson G P, Legler P M, Goldman E R. Evaluation of disulfide bond position to enhance the thermal stability of a highly stable single domain antibody. PLOS One (2014) 9:1-14. doi: 10.1371/journal.pone.0115405.
Zimmermann I, Egloff P, Hutter C A J, Arnold F M, Stohler P, Bocquet N, Hug M N, Huber S, Siegrist M, Hetemann L, et al. Synthetic single domain antibodies for the conformational trapping of membrane proteins. Elife (2018) 7:1-32. doi: 10.7554/eLife.34317
Zapata et al. Protein Eng. 8 (10): 1057-1062 (1995).
European Patent No. 404,097.
PCT Patent Publication WO 93/11161.
PCT Patent Publication WO 2020/176815.
U.S. Pat. No. 5,641,870.
U.S. Pat. No. 6,054,297.
U.S. Pat. No. 5,886,152.
U.S. Pat. No. 5,877,293.

What is claimed is:

1. A method of selecting a camelid nanobody from a library of camelid nanobody sequences collected from B cells from a camelid immunized with an antigen, the method comprising
(a) identifying a camelid nanobody that has at least one of the following features
(i) a phenylalanine (F) at position 42 (IMGT numbering);
(ii) a short hinge;
(iii) two or more cysteines in the nanobody sequence;
(iv) a glutamine (Q) at position 123 (IMGT numbering);
(v) low immunogenicity metric;
(vi) non-classic VHH derived from germline IGHV3 or a valine (V) at position 42 (IMGT numbering);
(vii) non-classic VHH derived from germline IGHV4 or an isoleucine (I) at position 42 (IMGT numbering);
(viii) a histidine (H), aspartic acid (D) or glutamic acid (E) in the CDR region;
(ix) a histidine (H), aspartic acid (D) or glutamic acid (E) in the first three amino acid residues, the FR2 region, or the first sixteen amino acid residues of the FR3 region of the nanobody sequence;
(x) a tyrosine (Y) at position 42 (IMGT numbering), and the nanobody having a loop, concave paratope structure configuration; or
(xi) a phenylalanine (F) at position 42 (IMGT numbering), and the nanobody having a convex paratope structure configuration; and
(b) measuring one or more biological activities of the nanobody identified in step (a).

2. The method of claim 1, wherein the low immunogenicity metric is measured by high similarity to human germlines using rarity score or percentage identity, or low 9-mer score.

3. The method of claim 1 or 2, wherein the selected nanobody has at least 2, 3, 4, 5, 6, 7, 8 or 9 of the features.

4. The method of any one of claims 1-3, wherein the biological activities comprise binding affinity, immunogenicity, thermostability, and pH sensitivity.

5. The method of any one of claims 1-4, wherein a nanobody with only two cysteines and an F at position 42 is selected.

7. The method of any one of claims 1-4, wherein the camelid nanobody in step (a) has at least one of the following features:
(i) a phenylalanine (F) at position 42 (IMGT numbering);
(ii) a short hinge; or
(iii) two cysteines within in the nanobody sequence; and
wherein the biological activity in step (b) is binding affinity.

8. The method of any one of claims 1-4, wherein the camelid nanobody in step (a) has at least one of the following features:
(i) low immunogenicity metric;
(ii) non-classic VHH derived from germline IGHV3 or a valine (V) at position 42 (IMGT numbering); or
(iii) non-classic VHH derived from germline IGHV4 or an isoleucine (I) at position 42 (IMGT numbering); and
wherein the biological activity in step (b) is immunogenicity in vitro or in vivo.

9. The method of any one of claims 1-4, wherein the camelid nanobody in step (a) has at least one of the following features:
(i) a glutamine (Q) at position 123 (IMGT numbering); or
(ii) four cysteines in the nanobody sequence; and
wherein the biological activity in step (b) is thermostability.

10. The method of any one of claims 1-4, wherein the camelid nanobody in step (a) has at least one of the following features:
(i) a histidine (H), aspartic acid (D) or glutamic acid (E) in the CDR region;
(ii) a histidine (H), aspartic acid (D) or glutamic acid (E) in the first three amino acid residues, the FR2 region, or the first sixteen amino acid residues of the FR3 region of the nanobody sequence; and
wherein the biological activity in step (b) is binding affinity at pH 6.0 versus pH 7.4, and wherein the nanobodies that are pH dependent are selected.

11. The method of any one of claims 1-4, wherein the camelid nanobody in step (a) has at least one of the following features:
(i) a tyrosine (Y) at position 42, and the nanobody having a loop, concave paratope structure configuration; or
(ii) a phenylalanine (F) at position 42, and the nanobody having a convex paratope structure configuration; and
wherein the biological activity in step (b) is binding affinity.

11. The method of any one of claims 1-10, wherein the library is an NGS library.

12. The method of claim 11, wherein the library data is processed by:
(a) grouping nanobody sequences in the libraries by CDR sequences (CDR groups), lineages and clusters; and
(b) generating an enrichment score for sequences, CDR groups, lineages and/or clusters by comparing their frequencies in NGS data after enrichment to those before enrichment or before and after immunization.

13. The method of any one of claims 1-12, wherein the selection procedure is repeated to optimize a sequence within the same lineage group of the selected antibodies.

14. The method of any one of claims 1-13, wherein the identifying procedure is repeated to optimize a sequence within the same lineage group of the selected antibodies.

15. The method of any one of claims 1-14, wherein the selected nanobody is expressed in prokaryotic cells.

16. The method of any one of claims 1-14, wherein the selected nanobody is expressed in eukaryotic cells.

17. The method of any one of claims 1-16, wherein binding affinity of the selected nanobody to the antigen is measured.

18. The method of any one of claims 1-17, wherein binding affinity of the selected nanobody to an epitope of the antigen is measured.

19. A method for generating a binder, wherein the binder binds to one or more nanobodies but does not substantially bind to an antibody having a leucine at position 123 (IMGT numbering) of the antibody sequence, comprising generating the binder that targets the FR4 region of an antibody having a glutamine at position 123 (IMGT numbering).

20. The method of claim 19, wherein the antibody having a leucine at position 123 (IMGT numbering) of the antibody sequence is a human antibody, a mouse antibody, or a rabbit antibody.

21. The method of claim 19 or 20, wherein the binder is an antibody or a binding fragment thereof.


SEQUENCE LISTING (28 sequences)

SEQ ID NO: 1 - protein, 17 aa, Vicugna pacos
MGWFRQAPGKEREFVAA

SEQ ID NO: 2 - protein, 17 aa, Vicugna pacos
MGWYRQAPGKQRELVAA

SEQ ID NO: 3 - protein, 17 aa, Vicugna pacos
MGWYRQAPGKQRELVAA

SEQ ID NO: 4 - protein, 17 aa, Vicugna pacos
MGWYRQAPGKERELVAA

SEQ ID NO: 5 - protein, 17 aa, Vicugna pacos
MGWYRQAPGKERELVAA

SEQ ID NO: 6 - protein, 17 aa, Vicugna pacos
MGWYRQAPGKERELVAA

SEQ ID NO: 7 - protein, 17 aa, Vicugna pacos
MGWFRQAPGKEREFVAA

SEQ ID NO: 8 - protein, 17 aa, Vicugna pacos
MGWFRQAPGKEREFVAA

SEQ ID NO: 9 - protein, 17 aa, Vicugna pacos
MGWFRQAPGKEREFVSC

SEQ ID NO: 10 - protein, 17 aa, Vicugna pacos
IGWFRQAPGKEREGVSC

SEQ ID NO: 11 - protein, 17 aa, Vicugna pacos
IGWFRQAPGKEREGVSC

SEQ ID NO: 12 - protein, 17 aa, Vicugna pacos
IGWFRQAPGKEREGVSC

SEQ ID NO: 13 - protein, 17 aa, Vicugna pacos
ISWFRQAPGKEREGVSC

SEQ ID NO: 14 - protein, 17 aa, Vicugna pacos
IGWFRQAPGKEREGVSC

SEQ ID NO: 15 - protein, 17 aa, Vicugna pacos
IGWFRQAPGKEREGVSC

SEQ ID NO: 16 - protein, 17 aa, Vicugna pacos
MSWVRQAPGKERELVAA

SEQ ID NO: 17 - protein, 17 aa, Vicugna pacos
MRWFRQAPGKEREWVSC

SEQ ID NO: 18 - protein, 29 aa, Vicugna pacos
EPKTPKPQPQPQPQPQPNPTTESKCPKCP

SEQ ID NO: 19 - protein, 15 aa, Vicugna pacos
AHHSEDPSSKCPKCP

SEQ ID NO: 20 - protein, 29 aa, Lama glama
EPKTPKPQPQPQPQPQPNPTTESKCPKCP

SEQ ID NO: 21 - protein, 15 aa, Lama glama
AHHSEDPSSKCPKCP

SEQ ID NO: 22 - protein, 35 aa, Camelus dromedarius
EPKIPQPQPKPQPQPQPQPKPQPKPEPECTCPKCP

SEQ ID NO: 23 - protein, 15 aa, Camelus dromedarius
AHHSEDPSSKCPKCP

SEQ ID NO: 24 - protein, 12 aa, Camelus dromedarius
GTNEVCKCPKCP

SEQ ID NO: 25 - protein, 35 aa, Camelus bactrianus; identical to SEQ ID NO: 22

SEQ ID NO: 26 - protein, 15 aa, Camelus bactrianus
AHHPEDPSSQCPKCP

SEQ ID NO: 27 - protein, 12 aa, Camelus bactrianus
GTNGGCKCPKCP

SEQ ID NO: 28 - protein, 12 aa, Camelus bactrianus
GTNEVCKCPKCP